SEQUENCE LISTING (27 sequences)

SEQ ID NO: 1 - protein, 31 aa, Artificial Sequence
  Synthetic
DAVDCALDPLSETKCTLKSFTVEKGIYQTSN

SEQ ID NO: 2 - protein, 75 aa, Artificial Sequence
  Synthetic
VCGPKKSTNLVKNKCVNFNFNGLTGTGVLTESNKKFLPFQQFGRDIADTTDAVRDPQTLEILDITPCSFGGVSVI

SEQ ID NO: 3 - protein, 40 aa, Artificial Sequence
  Synthetic
GTNTSNQVAVLYQDVNCTEVPVAIHADQLTPTWRVYSTGS

SEQ ID NO: 4 - protein, 18 aa, Artificial Sequence
  Synthetic
FSQILPDPSKPSKRSFIE

SEQ ID NO: 5 - protein, 22 aa, Artificial Sequence
  Synthetic
FGAGAALQIPFAMQMAYRFNGI

SEQ ID NO: 6 - protein, 21 aa, Artificial Sequence
  Synthetic
RPQGLPNNTASWFTALTQHGK

SEQ ID NO: 7 - protein, 20 aa, Artificial Sequence
  Synthetic
NNNAATVLQLPQGTTLPKGF

SEQ ID NO: 8 - protein, 47 aa, Artificial Sequence
  Synthetic
NKHIDAYKTFPPTEPKKDKKKKTDEAQPLPQRQKKQPTVTLLPAADM

SEQ ID NO: 9 - protein, 24 aa, Artificial Sequence
  Synthetic
MADSNGTITVEELKKLLEQWNLVI

SEQ ID NO: 10 - protein, 20 aa, Artificial Sequence
  Synthetic
PLLESELVIGAVILRGHLRI

SEQ ID NO: 11 - protein, 42 aa, Artificial Sequence
  Synthetic
MSDNGPQNQRNAPRITFGGPSDSTGSNQNGERSGARSKQRRP

SEQ ID NO: 12 - protein, 42 aa, Artificial Sequence
  Synthetic
QGLPNNTASWFTALTQHGKEDLKFPRGQGVPINTNSSPDDQI

SEQ ID NO: 13 - protein, 42 aa, Artificial Sequence
  Synthetic
GYYRRATRRIRGGDGKMKDLSPRWYFYYLGTGPEAGLPYGAN

SEQ ID NO: 14 - protein, 45 aa, Artificial Sequence
  Synthetic
KDGIIWVATEGALNTPKDHIGTRNPANNAAIVLQLPQGTTLPKKK

SEQ ID NO: 15 - protein, 42 aa, Artificial Sequence
  Synthetic
KGFYAEGSRGGSQASSRSSSRSRNSSRNSTPGSSRGTSPARM

SEQ ID NO: 16 - protein, 42 aa, Artificial Sequence
  Synthetic
AGNGGDAALALLLLDRLNQLESKMSGKGQQQQGQTVTKKSAA

SEQ ID NO: 17 - protein, 42 aa, Artificial Sequence
  Synthetic
EASKKPRQKRTATKAYNVTQAFGRRGPEQTQGNFGDQELIRQ

SEQ ID NO: 18 - protein, 45 aa, Artificial Sequence
  Synthetic
GTDYKHWPQIAQFAPSASAFFGMSRIGMEVTPSGTWLTYTGAKKK

SEQ ID NO: 19 - protein, 42 aa, Artificial Sequence
  Synthetic
IKLDDKDPNFKDQVILLNKHIDAYKTFPPTEPKKDKKKKADE

SEQ ID NO: 20 - protein, 41 aa, Artificial Sequence
  Synthetic
TQALPQRQKKQQTVTLLPAADLDDFSKQLQQSMSSADSTQA

SEQ ID NO: 21 - protein, 69 aa, Artificial Sequence
  Synthetic
SNNLDSKVGGNYNYLYRLFRKSNLKPFERDISTEIYQAGSTPCNGVEGFNCYFPLQSYGFQPTNGVGYQ

SEQ ID NO: 22 - protein, 26 aa, Artificial Sequence
  Synthetic
KKDKKKKADETQALPQRQKKQQTVTL

SEQ ID NO: 23 - protein, 419 aa, Artificial Sequence
  Synthetic
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 24 - protein, 1273 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 25 - protein, 425 aa, Artificial Sequence
  Synthetic
MHHHHHHSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 26 - protein, 679 aa, Artificial Sequence
  Synthetic
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAHHHHHHHHHH

SEQ ID NO: 27 - protein, 539 aa, Artificial Sequence
  Synthetic
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPAHHHHHHHHHH

(19) United States
(12) Patent Application Publication
(10) Pub. No.: US 2023/0213516 A1
(43) Pub. Date: Jul. 6, 2023

(54) IMMUNOASSAYS FOR DETECTION OF IMMUNOGLOBULINS AGAINST SARS COV-2 AND METHODS OF USE

(71) Applicant: Quidel Corporation, San Diego, CA (US)

(72) Inventors: Richard L. EGAN, Oceanside, CA (US); Werner KROLL, Wayland, CA (US); Jason MCCLURE, SAN DIEGO, CA (US); Robert REED, Carlsbad, CA (US); Jason SUN, San Diego, CA (US)

(21) Appl. No.: 17/996,735

(22) PCT Filed: Apr. 23, 2021
(86) PCT No.: PCT/US2021/028817

§ 371 Date: Oct. 20, 2022

Related U.S. Application Data
(60) Provisional application No. 63/166,849, filed on Mar. 26, 2021.
(60) Provisional application No. 63/041,029, filed on Jun. 18, 2020.
(60) Provisional application No. 63/015,412, filed on Apr. 24, 2020.

(51) Int. Cl. G01N 33/569; G01N 33/543

(52) U.S. Cl. CPC G01N 33/56983; G01N 33/54389; G01N 2469/20; G01N 2333/165

(57) ABSTRACT

Lateral flow immunoassays that reliably detect human antibodies, including IgG and/or IgM antibodies, specific for severe acute respiratory syndrome coronavirus 2 (SARS CoV-2) are described herein. Devices, methods and kits for analysis of samples, such as liquid blood, serum or plasma, for the presence of human antibodies, such as IgG and/or IgM antibodies, specific for SARS CoV-2 proteins, such as SARS CoV-2 N and/or S proteins are provided.

### CROSS-REFERENCE TO RELATED APPLICATIONS

This application claims the benefit of U.S. Provisional Application No. 63/166,849, filed Mar. 26, 2021; U.S. Provisional Application No. 63/041,029, filed Jun. 18, 2020; and U.S. Provisional Application No. 63/015,412, filed Apr. 24, 2020, each of which is incorporated herein by reference in its entirety.

### REFERENCE TO SEQUENCE LISTING, TABLE OR COMPUTER PROGRAM

Peptide sequences related to the present disclosure are provided in Table 1.

### TECHNICAL FIELD

The subject matter described herein relates to lateral flow immunoassays that reliably detect human IgG and/or IgM antibodies specific for severe acute respiratory syndrome coronavirus 2 (SARS CoV-2). Devices, methods and kits for analysis of samples for the presence of human IgG and/or IgM antibodies specific for SARS CoV-2 N proteins and/or SARS CoV-2 S are provided.

### BACKGROUND

Lateral flow immunoassays test devices have an extensive history of use in both the clinical and the home settings. These devices are used to test for a variety of analytes, such as hormones, proteins, urine or plasma components and the like. These devices generally comprise a lateral flow test strip, such as nitrocellulose or filter paper, a sample application area, test results area and an analyte specific binding reagent that is bound to some kind of detectable label, such as a colored particle, fluorescent or luminescent tag, or an enzyme detection system. The simplicity of such devices is a factor in maintaining their use in the marketplace, and additional tests for detection and differentiation of multiple analytes in a single sample are desired.

Severe acute respiratory syndrome coronavirus 2 (SARS CoV-2) is the virus strain that causes coronavirus disease 2019 (COVID-19), a respiratory illness. It is colloquially known as the coronavirus, and was previously referred to by its provisional name 2019 novel coronavirus (2019-nCoV). SARS CoV-2 is a positive-sense single-stranded RNA virus. It is contagious in humans, and the World Health Organization designated the 2019 pandemic of COVID-19 a Public Health Emergency of International Concern.

Like other known coronaviruses, SARS CoV-2 is an enveloped virus containing three outer structural proteins, namely the membrane (M), envelope (E), and spike (S) proteins. The nucleocapsid (N) protein together with the viral RNA genome presumably form a helical core located within the viral envelope. The SARS CoV-2 nucleocapsid (N) protein is a 423 amino-acid, predicted phospho-protein of 46 kDa that shares little homology with other members of the coronavirus family. SARS CoV-2 uses its spike glycoprotein (S), a main target for neutralization antibody, to bind its receptor, and mediate membrane fusion and virus entry. Each monomer of trimeric S protein is about 180 kDa, and contains two subunits, S1 and S2, mediating attachment and membrane fusion, respectively.

There remains a need for lateral flow immunoassays for detection of human antibodies against SARS CoV-2 that provide sensitive (decrease false negative results) and specific (decrease false positive results) sample analysis.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In one aspect, an immunoassay device for detection of an immunoglobulin for SARS CoV-2 is provided. The device comprises a sample receiving zone configured to receive a blood, plasma or serum sample; a detectable reagent comprising i) any SARS CoV-2 protein or peptide (such as, SARS CoV-2 N, S, S1, S2, M, and/or E proteins, peptides or fragments thereof) or ii) a non-human anti-human IgG, a non-human antihuman IgM antibody, or both; one or more capture zones comprising an test line comprising an immobilized SARS CoV-2 protein or peptide (such as, SARS CoV-2 N, S, S1, S2, M, and/or E proteins, peptides or fragments thereof); for example, an N-test line comprising an immobilized SARS CoV-2 N-protein or peptide; or, for example, an S-test line and an N-test line. In one embodiment, a sample deposited in the sample receiving zone is confirmed to comprise an immunoglobulin for SARS CoV-2 when the detectable reagent is detected at the S-test line, the N-test line, or both.

In another aspect, an immunoassay device for detection of an immunoglobulin for SARS CoV-2 is provided. The device comprises a sample receiving zone configured to receive a blood, plasma or serum sample; a detectable reagent comprising a SARS CoV-2 protein or peptide (such as, SARS CoV-2 N, S, S1, S2, M, and/or E proteins, peptides or fragments thereof), for example, a SARS CoV-2 S protein or peptide and a SARS CoV-2 N protein or peptide; and one or more captures zones comprising an S-test line comprising an immobilized SARS CoV-2 S protein or peptide; and/or an N-test line comprising an immobilized SARS CoV-2 N-protein or peptide, In one embodiment, a sample deposited in the sample receiving zone is confirmed to comprise an immunoglobulin for SARS CoV-2 when the detectable reagent is detected at the S-test line and the N-test line.

In another aspect, an immunoassay device for detection of an immunoglobulin for SARS CoV-2 is provided. The device comprises a sample receiving zone configured to receive a blood, plasma or serum sample; a detectable reagent comprising a non-human anti-human IgG and a non-human antihuman IgM antibody; one or more capture zones comprising an S-test line comprising an immobilized SARS CoV-2 S protein or peptide; and an N-test line comprising an immobilized SARS CoV-2 N-protein or peptide. A sample deposited in the sample receiving zone is confirmed to be from a subject with infection by SAR CoV-2 when the detectable reagent is detected at the S-test line and/or the N-test line.

In one embodiment, the device comprises two capture zones, a first capture zone comprising the S-test line and the N-test line for capture of the detectable reagent comprising a non-human anti-human IgG, and a second capture zone comprising a second S-test line and a second N-test line for capture of the detectable reagent comprising a non-human anti-human IgM.

In another aspect, an immunoassay device for detection of an IgG for SARS CoV-2 is provided. The device comprises a sample receiving zone configured to receive a blood, plasma or serum sample; a detectable reagent comprising a non-human anti-human IgG antibody; one or more capture zones comprising an S-test line comprising an immobilized SARS CoV-2 S protein or peptide; and an N-test line comprising an immobilized SARS CoV-2 N-protein or peptide. A sample deposited in the sample receiving zone is confirmed to comprise an IgG for SARS CoV-2 when the detectable reagent is detected at the S-test line and/or the N-test line.

In another aspect, an immunoassay device for detection of an IgM for SARS CoV-2 is provided. The device comprises a sample receiving zone configured to receive a blood, plasma or serum sample, a detectable reagent comprising a non-human anti-human IgM antibody; one or more capture zones comprising an S-test line comprising an immobilized SARS CoV-2 S protein or peptide; and an N-test line comprising an immobilized SARS CoV-2 N-protein or peptide. A sample deposited in the sample receiving zone is confirmed to comprise an IgM for SARS CoV-2 when the detectable reagent is detected at the S-test line and/or the N-test line.

In one embodiment, the one or more capture zones comprises a first capture zone and a second capture zone, the first capture zone comprising the N-test line in a first fluid flow path in communication with the sample receiving zone, and the second capture zone comprising the S-test line and in a second fluid flow path in communication with the sample receiving zone.

In one embodiment, the detectable reagent is deposited on the device in conjunction with a test sample.

In one embodiment, the detectable reagent is in a reagent zone downstream of the sample receiving zone.

In one embodiment, the SARS CoV-2 S protein or peptide and the SARS CoV-2 N-protein or peptide on the S-test line and the N-test line, respectively, is selected from the group of sequences in Table 1 or any sequence having 99%, 98%, 97%, 96%, 95%, 94%, 93%, 92%, 91%, 90%, 89%, 88%, 87%, 86% or 85% sequence identity thereto.

In one embodiment, the detectable reagent comprises a SARS CoV-2 S protein or peptide and a SARS CoV-2 N-protein or peptide selected from the group of sequences in Table 1 or any sequence having 99%, 98%, 97%, 96%, 95%, 94%, 93%, 92%, 91%, 90%, 89%, 88%, 87%, 86% or 85% sequence identity thereto.

In some embodiments, the SARS CoV-2 protein or peptide on, one or more test line, is any full length SARS CoV-2 protein, peptide or fragment thereof, such as full length N, S, S1, S2, M, and/or E proteins, peptides or fragments thereof or any sequence having 99%, 98%, 97%, 96%, 95%, 94%, 93%, 92%, 91%, 90%, 89%, 88%, 87%, 86% or 85% sequence identity thereto.

In some embodiments, the SARS CoV-2 mobilizable protein or peptide present in the sample label pad, is any full length SARS CoV-2 protein, peptide or fragment thereof, such as full length N, S, S1, S2, M, and/or E proteins, peptides or fragments thereof or any sequence having 99%, 98%, 97%, 96%, 95%, 94%, 93%, 92%, 91%, 90%, 89%, 88%, 87%, 86% or 85% sequence identity thereto.

In one embodiment, the SARS CoV-2 S protein or peptide and the SARS CoV-2 N-protein or peptide on, respectively, the S-test line and the N-test line, is, respectively, the full length SARS CoV-2 S protein and the full length SARS CoV-2 N-protein or any sequence having 99%, 98%, 97%, 96%, 95%, 94%, 93%, 92%, 91%, 90%, 89%, 88%, 87%, 86% or 85% sequence identity thereto.

In one embodiment, the one or more capture zones each comprise between 2-12, 3-15, 4-12, 6-12, 5-10, 6-10, 8-12, 8-10, 1, 2, 3, 4, 5, 6, 7, 8, 9, 10, 11, or 12 test lines, each test line in a discrete fluid flow path in fluid communication with the sample receiving zone.

In one embodiment, each test line in the one or more capture zones comprises a different immobilized SARS CoV-2 protein or peptide, such as full length N, S, S1, S2, M, and/or E proteins, peptides or fragments thereof.

In one embodiment, the device comprises 10 test lines, each test line in a discrete fluid flow path in fluid communication with the sample receiving zone.

In one embodiment, the detection reagent comprises a SARS CoV-2 S- or SARS CoV-2 N-protein or peptide, the at least one capture zone comprises the same SARS CoV-2 S- or SARS CoV-2 N-protein or peptides immobilized thereon.

In one embodiment, the detectable reagent comprises a detection moiety selected from a chelated lanthanide or metal.

In one embodiment, the chelated lanthanide is europium.

In one embodiment, the metal is gold.

In one embodiment, the sample receiving zone is configured to receive a blood sample and comprises a filtration member to separate red blood cells from the blood sample.

In one embodiment, the immunoglobulin is a human immunoglobulin specific for SARS CoV-2.

In one embodiment, the detectable reagent is a non-human anti-human IgG or IgM antibody, the antibody is a rabbit anti-human IgG or IgM antibody conjugated to a detectable moiety.

In one embodiment, the device further comprises a control line in the one or more capture zones.

In one embodiment, the N-test line comprises immobilized SARS CoV-2 N protein or peptide(s) only and does not comprise SARS CoV-2 S protein or peptide(s).

In one embodiment, the S-test line comprises immobilized SARS CoV-2 S protein or peptide(s) only and does not comprise SARS CoV-2 N protein or peptide(s).

In one embodiment, the assay is configured for insertion into and processing by an optical reader instrument.

In one embodiment, the optical reader instrument transmits results to a server for data collection and/or compilation purposes for surveillance of infection.

In one embodiment, the assay is configured for analysis by a personal smart device with a camera.

In one embodiment, the instrument or camera images the one or more captures zones in single image.

In another aspect, a kit is provided that is comprised of one or more immunoassay devices as described herein; optionally, a detection reagent; optionally, a blood collection device; and/or optionally, an auxiliary unit with an imaging system and electronics for transmitting an image wirelessly.

In another aspect, a method to distinguish an immunoglobulin for SARS CoV-2 as one from infection by SARS CoV-2 virus or one from a vaccination is provided. The method comprises detecting in a blood sample antibodies against a first SARS CoV-2 viral protein and a second SARS CoV-2 viral protein, such as the S protein of SARS CoV-2 virus, the M protein of SARS CoV-2 virus, the E protein of SARS-CoV-2 virus, and/or the N protein of SARS CoV-2 virus; and reporting one or more of:
i) the blood sample as being from an individual infected with SARS CoV-2 virus and/or previously vaccinated if antibodies against both the first protein (such as the N-protein) and the second protein (such as the S-protein) are detected;
ii) the blood sample as being from an individual with SARS CoV-2 virus if antibodies against the first protein (such as the N-protein) are detected and antibodies against the second protein (such as the S-protein) are not detected or are present but below a defined threshold;
iii) the blood sample as being from an individual previously treated with a vaccine against SARS CoV-2 virus if antibodies against the second protein (such as the S-protein) are detected and antibodies against the first protein (such as the N-protein) are not detected or are present but below a defined threshold;
iv) the blood sample as being from an individual with no immunoglobulin for SARS CoV-2 if antibodies against the second protein (such as the S-protein) and the first protein (such as the N-protein) are not detected; or
v) invalid result if a control line has no detectable control reagent.

In another aspect, a method for determining type or presence of immunity against SARS CoV-2 in a subject and/or for determining if a subject needs to be vaccinated against SARS CoV-2 is provided. The method comprises providing an immunoassay test device or a kit as described herein, and placing or instructing to place a sample, such as a blood, serum or plasma sample from the subject on the test device; inspecting or instructing to inspect the N-test line and the S-test line in the one or more capture zones of the device; and reporting or having reported based on the inspecting one of the following:
i) the subject was infected with SARS CoV-2 virus and/or previously vaccinated if detectable reagent at both the N-test line and the S-test line is present;
ii) the subject was or is infected with SARS CoV-2 virus if detectable reagent at the N-test line is present and detectable reagent at the S-test line is not present or are present but below a defined threshold;
iii) the subject was previously treated with a vaccine against SARS CoV-2 virus if detectable reagent is present at the S-test line is present and detectable reagent at the N-test line is not present or are present but below a defined threshold;
iv) the subject has no immunity against SARS CoV-2 if no detectable reagent is present at the S- or N-test lines; or
v) invalid result if a control line has no detectable control reagent.

In another aspect, a method for determining type or presence of immunity against SARS CoV-2 in a subject and/or for determining if a subject needs to be vaccinated against SARS CoV-2 is provided. The method comprises providing an immunoassay test device or a kit as described herein, and placing or instructing to place a sample, such as a blood, serum or plasma sample from the subject on the test device; inspecting or instructing to inspect the first test line (such as an N-test line) and the second test line (such as an S-test line) in the one or more capture zones of the device; and reporting or having reported based on the inspecting one of the following:
i) the subject was infected with SARS CoV-2 virus and/or previously vaccinated if detectable reagent at both the first and the second test lines is present;
ii) the subject was or is infected with SARS CoV-2 virus if detectable reagent at the first test line is present and detectable reagent at the second test line is not present or are present but below a defined threshold;
iii) the subject was previously treated with a vaccine against SARS CoV-2 virus if detectable reagent is present at the second test line is present and detectable reagent at the first test line is not present or are present but below a defined threshold;
iv) the subject has no immunity against SARS CoV-2 if no detectable reagent is present at the either of the first or the second test lines; or
v) invalid result if a control line has no detectable control reagent.

In one embodiment, the first test line comprises a protein or antigen from the N, E, S, or M protein of SARS CoV-2. In one embodiment, the second test line comprises a protein or antigen from the N, E, S, or M protein of SARS CoV-2.

In one embodiment, detecting or inspecting is via an instrument with a scanning photo diode or a camera that takes an image of the one or more capture zones.

In one embodiment, the camera is a personal smart phone or device.

In one embodiment, the scanning photo diode comprises two or more photodiodes to interrogate separately the one or more capture zones or different test lines.

In one embodiment, the image is transmitted to a server for processing to determine i)-v) for the reporting.

In one embodiment, results i)-v) is/are transmitted to a server for compilation and/or reporting with additional blood samples, for surveillance of SARS CoV-2 infection.

In another aspect, an immunoassay device for detection of an IgG for SARS CoV-2 is provided. In one embodiment, the immunoassay includes a sample receiving zone configured to receive a blood, plasma or serum sample, a detectable reagent comprising a non-human anti-human IgG antibody and one or more capture zones. In one embodiment, the capture zones include an S1-test line comprising an immobilized SARS CoV-2 S1 protein or peptide, an S2-test line comprising an immobilized SARS CoV-2 S2 protein or peptide, and an N-test line comprising an immobilized SARS CoV-2 N-protein or peptide. In one embodiment, a sample deposited in the sample receiving zone is confirmed to comprise an IgG for SARS CoV-2 when the detectable reagent is detected at the S1-test line, S2-test line and/or the N-test line.

In another embodiment, the one or more capture zones further comprises a reference line. In another embodiment, the one or more capture zones comprises a first capture zone and a second capture zone, the first capture zone comprising the S1-test line and the S2-test line in a first fluid flow path in communication with the sample receiving zone, and the second capture zone comprising the N-test line and the reference line in a second fluid flow path in communication with the sample receiving zone. In yet another embodiment, the one or more capture zones comprises a first capture zone and a second capture zone, the first capture zone comprising the S1-test line and the N-test line in a first fluid flow path in communication with the sample receiving zone, and the second capture zone comprising the S2-test line and the reference line in a second fluid flow path in communication with the sample receiving zone. In another embodiment, the one or more capture zones comprises a first capture zone and a second capture zone, the first capture zone comprising the S2-test line and the N-test line in a first fluid flow path in communication with the sample receiving zone, and the second capture zone comprising the S1-test line and the reference line in a second fluid flow path in communication with the sample receiving zone.

In some embodiments, a sample deposited in the sample receiving zone is confirmed to be from a SARS CoV-2 vaccinated individual when the detectable reagent is detected at the S1-test line and not at the N-test line. In other embodiments, a sample deposited in the sample receiving zone is confirmed to be from an individual with a prior natural SARS CoV-2 infection when the detectable reagent is detected at least at the N-test line.

In addition to the exemplary aspects and embodiments described above, further aspects and embodiments will become apparent by reference to the drawings and by study of the following descriptions.

Additional embodiments of the present devices, methods, kits and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF THE DRAWINGS

FIG. 1A-B illustrates a top view of bidirectional test devices (1A, IgG and 1, IgM) with a centrally located sample receiving zone and bilateral fluid flow paths. Positions of optical windows for inspection of the test lines are also shown. In this exemplary configuration, cassette 1 detects human IgG antibodies specific for SARS CoV-2 N and S proteins, while cassette 2 detects human IgM antibodies specific for SARS CoV-2 N and S proteins.

FIG. 2 illustrates a top view of a bidirectional test device with a centrally located sample receiving zone and bilateral fluid flow paths. Positions of optical windows for inspection of the test lines are also shown. In this exemplary configuration, the cassette detects human IgG antibodies for SARS CoV-2 N and S proteins and human IgM antibodies for SARS CoV-2 N and S proteins.

FIG. 3 illustrates a top view of a unidirectional test device with a plurality of parallel flow paths. In this exemplary configuration, the cassette detects either human IgG or IgM antibodies for multiple SARS CoV-2 N peptides and full length N protein and/or SARS CoV-2 S peptides and/or full length S protein.

FIG. 4 illustrates a top view of a bidirectional test device with centrally located sample receiving zone and a plurality of parallel flow paths. In this exemplary configuration, the cassette detects human IgG and IgM antibodies for multiple SARS CoV-2 N and S peptides and full length proteins.

FIG. 5A-B illustrates a top view of bidirectional test devices (5A: cassette 1, IgG and 5B, cassette 2, IgM) with a centrally located sample receiving zone and bilateral fluid flow paths. Positions of optical windows for inspection of the test lines are also shown. In this exemplary configuration, cassette 1 detects human IgG antibodies specific for SARS CoV-2 N, S1, and S2 protein, while cassette 2 detects human IgM antibodies specific for SARS CoV-2 N, S1 and S2 proteins.

FIG. 6A-C provide semi-quantitative signal to cutoff (S/CO) results for S1 (6A), S2 (6B) and N (6C) from finger stick whole blood, for subjects without prior SARS CoV-2 exposure plotted over time.

FIG. 7A-C provide semi-quantitative signal to cutoff (S/CO) results for S1 (7A), S2 (7B) and N (7C) detection in samples from individuals with no prior SARS COV-2 infection compared to samples from individuals with prior natural SARS COV-2 infection plotted over time.

FIG. 8A-D provide a graphical representation of statistical analysis for detection of S1 (8A, Passing Bablok regression fit), S2 (8D, Passing Bablok regression fit), and N (8C, Passing Bablok regression fit; and 8D, Passing Bablok regression fit (N S/CO>=1 IgG)) antibodies from finger stick whole blood, venous whole blood, serum, and plasma samples.

TABLE 1
BRIEF DESCRIPTION OF THE SEQUENCES

| SEQ ID NO | SEQUENCE | MODIFICATION |
|---|---|---|
| #1 S(287-317) | DAVDCALDPLSETKCTLKSFTVEKGIYQTSN | N-Terminal: |
| | | Biotin-miniPEG |
| #2 S(524-598) | VCGPKKSTNLVKNKCVNFNFNGLTGTGVLTESNKKFLPFQ | N-Terminal: |
| | QFGRDIADTTDAVRDPQTLEILDITPCSFGGVSVI | Biotin-miniPEG |
| #3 S(601-640) | GTNTSNQVAVLYQDVNCTEVPVAIHADQLTPTWRVYSTGS | N-Terminal: |
| | | Biotin-miniPEG |
| #4 S(802-819) | FSQILPDPSKPSKRSFIE | N-Terminal: |
| | | Biotin-miniPEG |
| #5 S(888-909) | FGAGAALQIPFAMQMAYRFNGI | N-Terminal: |
| | | Biotin-miniPEG |
| #6 N(42-62) | RPQGLPNNTASWFTALTQHGK | N-Terminal: |
| | | Biotin-miniPEG |
| #7 N(153-172) | NNNAATVLQLPQGTTLPKGF | N-Terminal: |
| | | Biotin-miniPEG |
| #8 N(355-401) | NKHIDAYKTFPPTEPKKDKKKKTDEAQPLPQRQKKQPTVTL | N-Terminal: |
| | LPAADM | Biotin-miniPEG |
| #9 M(1-24) | MADSNGTITVEELKKLLEQWNLVI | N-Terminal: |
| | | Biotin-miniPEG |
| #10 M(132-151) | PLLESELVIGAVILRGHLRI | N-Terminal: |
| | | Biotin-miniPEG |
| #11 N Peptide 1 | MSDNGPQNQRNAPRITFGG PSDSTGSNQNGERSGARS | N-Terminal: |
| | KQRRP | Biotin-miniPEG |
| #12 N Peptide 2 | QGLPNNTASWFTALTQHGKEDLKFPRGQGVPINTNSSP | N-Terminal: |
| | DDQI | Biotin-miniPEG |
| #13 N Peptide 3 | GYYRRATRRIRGGDGKMKD LSPRWYFYYLGTGPEAGLP | N-Terminal: |
| | YGAN | Biotin-miniPEG |
| #14 N Peptide 4 | KDGIIWVATEGALNTPKDHIGTRNPANNAAIVLQLPQGTT | N-Terminal: |
| | LPKKK | Biotin-miniPEG |
| #15 N Peptide 5 | KGFYAEGSRGGSQASSRSS SRSRNSSRNSTPGSSRGTS | N-Terminal: |
| | PARM | Biotin-miniPEG |
| #16 N Peptide 6 | AGNGGDAALALLLLDRLNQ LESKMSGKGQQQQGQTVT | N-Terminal: |
| | KKSAA | Biotin-miniPEG |
| #17 N Peptide 7 | EASKKPRQKRTATKAYNVT QAFGRRGPEQTQGNFGDQ | N-Terminal: |
| | ELIRQ | Biotin-miniPEG |
| #18 N Peptide 8 | GTDYKHWPQIAQFAPSASA FFGMSRIGMEVTPSGTWLT | N-Terminal: |
| | YTGAKKK | Biotin-miniPEG |
| #19 N Peptide 9 | IKLDDKDPNFKDQVILLNKHI DAYKTFPPTEPKKDKKKKA | N-Terminal: |
| | DE | Biotin-miniPEG |
| #20 Peptide 10 | TQALPQRQKKQQTVTLLPAADLDDFSKQLQQSMSSADS | N-Terminal: |
| | TQA | Biotin-miniPEG |
| #21 S (437-506) | SNNLDSKVGG NYNYLYRLFR KSNLKPFERD ISTEIYQAGS | N-Terminal: |
| | TPCNGVEGFN CYFPLQSYGF QPTNGVGYQ | Biotin-miniPEG |
| #22 N(370-395) | KKDKKKKADETQALPQRQKKQQTVTL | N-Terminal: |
| | | Biotin-miniPEG |
| #23 Full length | MSDNGPQNQRNAPRITFGGPSDSTGSNQNGERSGARSKQRR | N/A, full length |
| SARS CoV-2 N- | PQGLPNNTASWFTALTQHGKEDLKFPRGQ | N protein |
| protein (NP) | GVPINTNSSPDDQIGYYRRATRRIRGGDGKMKDLSPRWYFY | sequence |
| (YP_009724397. | YLGTGPEAGLPYGANKDGIIWVATEGALN | |
| 2) | TPKDHIGTRNPANNAAIVLQLPQGTTLPKGFYAEGSRGGSQ | |
| | ASSRSSSRSRNSSRNSTPGSSRGTSPARM | |
| | AGNGGDAALALLLLDRLNQLESKMSGKGQQQQGQTVTKK | |
| | SAAEASKKPRQKRTATKAYNVTQAFGRRGPE | |
| | QTQGNFGDQELIRQGTDYKHWPQIAQFAPSASAFFGMSRIG | |
| | MEVTPSGTWLTYTGAIKLDDKDPNFKDQV | |
| | ILLNKHIDAYKTFPPTEPKKDKKKKADETQALPQRQKKQQT | |
| | VTLLPAADLDDFSKQLQQSMSSADSTQA | |
| #24 Full length | MFVFLVLLPLVSSQCVNLTTRTQLPPAYTNSFTRGVYYPDK | N/A, full length |
| SARS CoV-2 S- | VFRSSVLHSTQDLFLPFFSNVTWFHAIHV | S protein |
| protein (SP) | SGTNGTKRFDNPVLPFNDGVYFASTEKSNIIRGWIFGTTLDS | sequence |
| (YP_009724390.1) | KTQSLLIVNNATNVVIKVCEFQFCNDPF | |
| | LGVYYHKNNKSWMESEFRVYSSANNCTFEYVSQPFLMDLE | |
| | GKQGNFKNLREFVFKNIDGYFKIYSKHTPI | |
| | NLVRDLPQGFSALEPLVDLPIGINITRFQTLLALHRSYLTPGD | |
| | SSSGWTAGAAAYYVGYLQPRTFLLKYN | |
| | ENGTITDAVDCALDPLSETKCTLKSFTVEKGIYQTSNFRVQP | |
| | TESIVRFPNITNLCPFGEVFNATRFASV | |
| | YAWNRKRISNCVADYSVLYNSASFSTFKCYGVSPTKLNDL | |
| | CFTNVYADSFVIRGDEVRQIAPGQTGKIAD | |
| | YNYKLPDDFTGCVIAWNSNNLDSKVGGNYNYLYRLFRKSN | |
| | LKPFERDISTEIYQAGSTPCNGVEGFNCYF | |
| | PLQSYGFQPTNGVGYQPYRVVVLSFELLHAPATVCGPKKST | |
| | NLVKNKCVNFNFNGLTGTGVLTESNKKFL | |
| | PFQQFGRDIADTTDAVRDPQTLEILDITPCSFGGVSVITPGTN | |
| | TSNQVAVLYQDVNCTEVPVAIHADQLT | |
| | PTWRVYSTGSNVFQTRAGCLIGAEHVNNSYECDIPIGAGICA | |
| | SYQTQTNSPRRARSVASQSIIAYTMSLG | |
| | AENSVAYSNNSIAIPTNFTISVTTEILPVSMTKTSVDCTMYIC | |
| | GDSTECSNLLLQYGSFCTQLNRALTGI | |
| | AVEQDKNTQEVFAQVKQIYKTPPIKDFGGFNFSQILPDPSKP | |
| | SKRSFIEDLLFNKVTLADAGFIKQYGDC | |
| | LGDIAARDLICAQKFNGLTVLPPLLTDEMIAQYTSALLAGTI | |
| | TSGWTFGAGAALQIPFAMQMAYRFNGIG | |
| | VTQNVLYENQKLIANQFNSAIGKIQDSLSSTASALGKLQDV | |
| | VNQNAQALNTLVKQLSSNFGAISSVLNDI | |
| | LSRLDKVEAEVQIDRLITGRLQSLQTYVTQQLIRAAEIRASA | |
| | NLAATKMSECVLGQSKRVDFCGKGYHLM | |
| | SFPQSAPHGVVFLHVTYVPAQEKNFTTAPAICHDGKAHFPR | |
| | EGVFVSNGTHWFVTQRNFYEPQIITTDNT | |
| | FVSGNCDVVIGIVNNTVYDPLQPELDSFKEELDKYFKNHTS | |
| | PDVDLGDISGINASVVNIQKEIDRLNEVA | |
| | KNLNESLIDLQELGKYEQYIKWPWYIWLGFIAGLIAIVMVTI | |
| | MLCCMTSCCSCLKGCCSCGSCCKFDEDD | |
| | SEPVLKGVKLHYT | |
| #25 N (1-419) | MHHHHHHSDNGPQNQRNAPRITFGGPSDSTGSNQNGERSG | N/A |
| | ARSKQRRPQGLPNNTASWFTALTQHGKEDLKFPRGQGVPI | |
| | NTNSSPDDQIGYYRRATRRIRGGDGKMKDLSPRWYFYYLG | |
| | TGPEAGLPYGANKDGIIWVATEGALN | |
| | TPKDHIGTRNPANNAAIVLQLPQGTTLPKGFYAEGSRGGSQ | |
| | ASSRSSSRSRNSSRNSTPGSSRGTSPARM | |
| | AGNGGDAALALLLLDRLNQLESKMSGKGQQQQGQTVTKK | |
| | SAAEASKKPRQKRTATKAYNVTQAFGRRGPE | |
| | QTQGNFGDQELIRQGTDYKHWPQIAQFAPSASAFFGMSRIG | |
| | MEVTPSGTWLTYTGAIKLDDKDPNFKDQV | |
| | ILLNKHIDAYKTFPPTEPKKDKKKKADETQALPQRQKKQQT | |
| | VTLLPAADLDDFSKQLQQSMSSADSTQA | |
| #26 S1(16-684) | VNLTTRTQLPPAYTNSFTRGVYYPDKVFRSSVLHSTQDLFL | N/A |
| | PFFSNVTWFHAIHVSGTNGTKRFDNPVLPFNDGVYFASTEK | |
| | SNIIRGWIFGTTLDSKTQSLLIVNNATNVVIKVCEFQFCNDPF | |
| | LGVYYHKNNKSWMESEFRVYSSANNCTFEYVSQPFLMDLE | |
| | GKQGNFKNLREFVFKNIDGYFKIYSKHTPINLVRDLPQGFSA | |
| | LEPLVDLPIGINITRFQTLLALHRSYLTPGDSSSGWTAGAAA | |
| | YYVGYLQPRTFLLKYN | |
| | ENGTITDAVDCALDPLSETKCTLKSFTVEKGIYQTSNFRVQP | |
| | TESIVRFPNITNLCPFGEVFNATRFASVYAWNRKRISNCVAD | |
| | YSVLYNSASFSTFKCYGVSPTKLNDLCFTNVYADSFVIRGD | |
| | EVRQIAPGQTGKIADYNYKLPDDFTGCVIAWNSNNLDSKV | |
| | GGNYNYLYRLFRKSNLKPFERDISTEIYQAGSTPCNGVEGF | |
| | NCYFPLQSYGFQPTNGVGYQPYRVVVLSFELLHAPATVCGP | |
| | KKSTNLVKNKCVNFNFNGLTGTGVLTESNKKFLPFQQFGR | |
| | DIADTTDAVRDPQTLEILDITPCSFGGVSVITPGTNTSNQVA | |
| | VLYQDVNCTEVPVAIHADQLTPTWRVYSTGSNVFQTRAGC | |
| | LIGAEHVNNSYECDIPIGAGICASYQTQTNSPRRAHHHHHH | |
| | HHHH | |
| #27 S2 (686- | SVASQSIIAYTMSLGAENSVAYSNNSIAIPTNFTISVTTEILPV | N/A |
| 1213) | SMTKTSVDCTMYICGDSTECSNLLLQYGSFCTQLNRALTGI | |
| | AVEQDKNTQEVFAQVKQIYKTPPIKDFGGFNFSQILPDPSKP | |
| | SKRSFIEDLLFNKVTLADAGFIKQYGDCLGDIAARDLICAQK | |
| | FNGLTVLPPLLTDEMIAQYTSALLAGTITSGWTFGAGAALQI | |
| | PFAMQMAYRFNGIGVTQNVLYENQKLIANQFNSAIGKIQDS | |
| | LSSTASALGKLQDVVNQNAQALNTLVKQLSSNFGAISSVLN | |
| | DILSRLDKVEAEVQIDRLITGRLQSLQTYVTQQLIRAAEIRAS | |
| | ANLAATKMSECVLGQSKRVDFCGKGYHLMSFPQSAPHGV | |
| | VFLHVTYVPAQEKNFTTAPAICHDGKAHFPREGVFVSNGTH | |
| | WFVTQRNFYEPQIITTDNTFVSGNCDVVIGIVNNTVYDPLQP | |
| | ELDSFKEELDKYFKNHTSPDVDLGDISGINASVVNIQKEIDR | |
| | LNEVAKNLNESLIDLQELGKYEQYIKWPAHHHHHHHHHH | |

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

A “lateral flow immunoassay” or “test strip” can include one or more bibulous or non-bibulous materials. If a test strip comprises more than one material, the one or more materials are preferably in fluid communication. One material of a test strip may be overlaid on another material of the test strip, such as for example, filter paper overlaid on nitrocellulose. Alternatively or in addition, a test strip may include a region comprising one or more materials followed by a region comprising one or more different materials. In this case, the regions are in fluid communication and may or may not partially overlap one another. Suitable materials for test strips include, but are not limited to, materials derived from cellulose, such as filter paper, chromatographic paper, nitrocellulose, and cellulose acetate, as well as materials made of glass fibers, nylon, dacron, PVC, polyacrylamide, cross-linked dextran, agarose, polyacrylate, ceramic materials, and the like. The material or materials of the test strip may optionally be treated to modify their capillary flow characteristics or the characteristics of the applied sample. For example, the sample application region of the test strip may be treated with buffers to correct the pH or specific gravity of an applied urine sample, to ensure optimal test conditions.

“Sample” is any material to be tested for the presence or amount of an analyte of interest. Preferably, a sample is a fluid sample, preferably a liquid sample. Examples of liquid samples that may be tested using a test device include bodily fluids including blood, serum, plasma, saliva, urine, ocular fluid, semen, sputum, nasal discharge and spinal fluid. For example, a sample for testing on a disclosed device may comprise liquid serum or plasma from a venous blood source where the serum or plasma has been separated from whole blood by centrifugation. In other cases, a sample may be liquid plasma from a finger prick that has been separated from whole blood by a blood-plasma separator. Other sample examples include liquid plasm from a finger prick that has been separated from whole blood by the lateral flow device.

“Analyte” is any substance of interest potentially present in a sample. For example, analytes include hormones, proteins, urine or plasma components and the like. Other examples of analytes include antibodies. For example, human IgG and IgM antibodies specific for SARS CoV-2 are particular analytes of interest that may be present in a sample tested by the present devices, methods and kits. The skilled artisan also appreciates that other immunoglobulins, including other human immunoglobulins such as IgA, are suitable analytes for analysis by lateral flow immunoassays.

“Reaction partner” refers to a substance, such as an antibody or antigen, in a lateral flow immunoassay that binds to an analyte of interest, such as human immunoglobulins. Peptide sequences related to the present disclosure and that may comprise reaction partners are provided in Table 1. For example, reaction partners for analytes such as human IgG and IgM antibodies specific for SARS CoV-2 include SARS CoV-2 proteins, peptides, such as SARS CoV-2 membrane (M), envelope (E), spike (S, including S1 and S2 subunits), and nucleocapsid (N) proteins. The nucleocapsid (N) protein together with the viral RNA genome presumably form a helical core located within the viral envelope. The SARS CoV-2 nucleocapsid (N) protein is a 423 amino-acid, predicted phospho-protein of 46 kDa that shares little homology with other members of the coronavirus family. SARS CoV-2 uses its spike glycoprotein (S), a main target for neutralization antibody, to bind its receptor, and mediate membrane fusion and virus entry. Each monomer of trimeric S protein is about 180 kDa, and contains two subunits, S1 and S2, mediating attachment and membrane fusion, respectively.

SARS CoV-2 N-proteins and peptides include full length N-protein, and specific epitopes of full length N-protein. Proteins and peptides may be selected as reaction partners based on sequences and/or immunogenicity analysis represented by respective peptides. Peptides represented by a SARS CoV-2 N-protein epitope map based on segmenting full length N-protein into segments of 10-100 amino acid lengths may also provide reaction partners for some embodiments of the present technology. Other examples of reaction partners include full length SARS CoV-2 S-protein, at least one specific epitope of full length SARS CoV-2 S-protein based on sequence and/or immunogenicity analysis represented by respective peptides. Peptides represented by SARS CoV-2 S-protein epitope map based on segmenting full length S-protein in segments of 10-100 amino acid lengths may also provide reaction partners for some embodiments of the present technology.

Particular proteins and peptides related to SARS CoV-2 N and S proteins are presented in Table 1. These peptides may comprise antigens and/or epitopes for human antibodies that are specific for SARS CoV-2 and can accordingly be used as components in the devices, methods and kits described herein for the detection of such antibodies on with lateral flow immunoassays.

“Readout” refers to the manner in which the test and/or reference lines may be interrogated for presence or absences of a positive signal. Positions of optical windows on the devices allow for inspection of the test and reference by either an instrument or by manual inspection. For example, an objective readout can be made by an optical reader instrument in order to allow for quantitative analysis that can be reduced to qualitative interpretation (signal over cutoff). Detection of a positive signal at a test line indicates the presence of an analyte of interest in the tested sample.

Test strips or devices also, optionally, comprise a control line or zone and/or a reference line or zone. If present, such zones or lines comprise an immobilized species with binding affinity for a detectable moiety deposited on or formed in a flow path on the device upstream of the control or reference line or zone. An optional reference or control line or zone may be positioned downstream of a test zone and comprises a binding member of a binding pair independent from the analyte of interest. Detection of a positive signal at a control/reference line indicates that the test strip performed properly, while absence of a signal at a control/reference line indicates that the test strip may not have functioned properly.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. Devices

In a first aspect, a device for determining presence, or absence, of antibodies against SARS CoV-2 is provided. Various embodiments of the device will be described with reference to certain drawing figures.

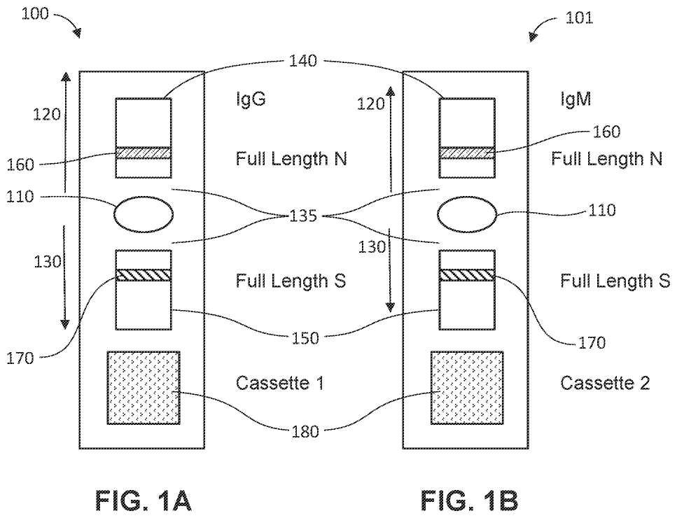

A first embodiment of a device is shown in FIGS. 1A and 1B which illustrates a top view of bidirectional test devices 100 (IgG) and 101 (IgM) with a centrally located sample receiving zones 110 and bilateral fluid flow paths 120 and 130. Positions of optical windows 140 and 150 for inspection of the test lines 160 and 170 are also shown. This embodiment also includes a barcode 180 for unique identification of the device. In this exemplary “antibody capture” configuration, cassette 1 detects human IgG antibodies specific for SARS CoV-2 N and S proteins, while cassette 2 detects human IgM antibodies specific for SARS CoV-2 N and S proteins. Accordingly, analysis of a sample by both cassettes 1 and 2 will determine whether the sample contains any, or all, of human IgG specific for SARS CoV-2 N-protein, human IgG specific for SARS CoV-2 S-protein, human IgM specific for SARS CoV-2 N-protein, and human IgM specific for SARS CoV-2 S-protein.

In this embodiment, specificity for human IgG or IgM is achieved by the inclusion of a non-human anti human IgG or IgM antibody in the device. The non-human anti human IgG or IgM antibody can be included, in a mobilizable fashion, in a sample pad (not shown) wherein the sample pad is located in a sample label zone 135 downstream of the sample receiving zone 110 and upstream of the test lines 160 and 170. The non-human anti human IgG or IgM antibody can also be present in a reaction mixture or excipient mixture added to the sample prior to its addition to the sample receiving zone. In some embodiments, the non-human anti human IgG or IgM is a rabbit antibody specific for human IgG or IgM (rb-α-IgGh or rb-α-IgMh). The non-human anti human IgG or IgM antibody may also include a detectable label for visualization purposes. For example, the non-human anti human antibody may include a detectable bead, such as a europium bead or a gold particle. Accordingly, the non-human anti human antibody, in some embodiments, is a rabbit antibody, specific to human IgG or IgM, conjugated with a detectable label, such as a europium bead or a gold particle. The detectable non-human anti human IgG and/or IgM antibody is referred to herein as a detectable reagent.

Also in this embodiment, specificity for SARS CoV-2 N or S-protein is conferred by the presence of SARS CoV-2 N or S-proteins or peptides immobilized on the test lines 160 and 170 of the device. For example, detection of a signal on the top test line (160) indicates the sample contains human antibodies specific for SARS CoV-2 N-protein while detection of a signal on the bottom test line (170) indicates the sample contains human antibodies specific to SARS CoV-2 S protein.

The components of the device provided in FIG. 1 exemplify one possible arrangement of the provided reagents and devices described herein. However, this arrangement is not to be construed as limiting, only as exemplary. Alternative arrangements of the reagents, some of which are described below, may be apparent to the skilled artisan and are encompassed by this disclosure.

For example, in FIG. 1, the positions of the immobilized SARS CoV-2 N and S proteins could be swapped. Alternatively, cassette 1 could be made specific for IgM with cassette 2 being specific for IgG without interfering with the performance of the device. Also, the mobilizable non-human antibodies specific for human IgG or IgM could instead be immobilized on the test lines (160 and 170) while the SARS CoV-2 N and/or S-proteins and peptides could be labeled, mobilizable and present on a sample pad in the sample label zone 135.

In addition, in FIG. 1, SARS CoV-2 N and/or S proteins and peptides could serve as both the labeled, mobilizable element and the capture agent immobilized at the test lines (160 and 170) in a double antigen “sandwich” assay consistent with the device provided in FIG. 1. In this arrangement, any human or non-human antibody in a test sample that is specific to SAR COV-2 N and/or S protein would first bind the labeled, mobilizable N or S protein or peptide to form a complex. The detectable labeled, mobilizable N or S protein or peptides are referred to herein as detectable reagents. In an embodiment, the detectable reagent is deposited in a sample label zone 135 and one bound to any IgG or IgM present in the test sample, the complex would then move downstream where it would be immobilized and visualized by binding another N or S protein or peptide that is immobilized on the test strip on the test lines (160 and 170). This “sandwich” arrangement demonstrates another alternative configuration of the devices described herein. In another embodiment, the detectable reagent is provided in a vial or container separate from the immunoassay device, such as in a reagent mixture or excipient mixture, that is combined with the test sample prior to its placement on the sample receiving zone.

The potential rearrangements would not interfere with the performance of the device and would still permit analysis of a sample for the presence of human IgG and/or IgM antibodies specific for SARS CoV-2 N and/or S-proteins and peptides. Accordingly, these alternative arrangements of components are included, either explicitly or implicitly, in this disclosure.

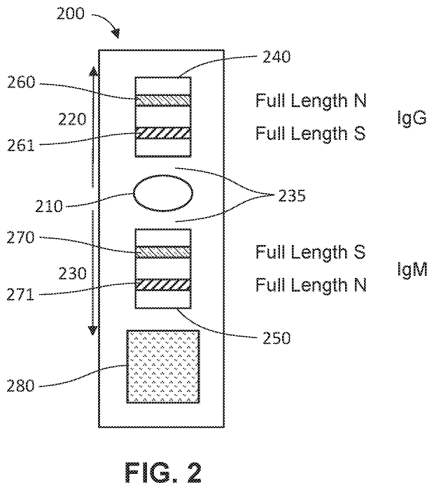

Another embodiment of the present disclosure is presented in FIG. 2 which illustrates a top view of a bidirectional test device 200 with a centrally located sample receiving zone 210 and bilateral fluid flow paths 220 and 230. Positions of optical windows 240 and 250 for inspection of the test lines 260, 261 and 270, 271 are also shown. This embodiment also includes a barcode 280 for unique identification of the device. In this exemplary configuration, the cassette detects human IgG antibodies for SARS CoV-2 N and S proteins and human IgM antibodies for SARS CoV-2 N and S proteins.

One exemplary configuration of the device shown in FIG. 2 includes an “antibody capture” approach wherein the sample is contacted with a labeled, non-human antibody specific for human IgG or IgM antibodies. Combination of the sample with the labeled, non-human antibody can be achieved by mixing the components prior to adding to the sample receiving zone 210, or can occur on the test strip when mobilizable, labeled non-human antibodies specific for human IgG or IgM are present on a sample pad in the sample label zone 235. Complexes of the mobilizable, labeled non-human antibodies bound to human IgG or IgM then travel down the strip where they are interrogated for specificity to SARS CoV-2 N or S-proteins at the test lines (260, 261 and 270, 271) comprising immobilized SARS CoV-2 N or S-proteins or peptides. If the sample contains human IgG or IgM antibodies specific for SARS CoV-2 N or S-proteins, then a signal will develop on the corresponding test line(s) (260, 261 and 270, 271).

As described above for FIG. 1, the components of the device shown in FIG. 2 may be arranged in different orders and configurations without interfering with the performance of the device. For example, the positions of the non-human antibodies specific for human IgG and IgM could be switched. Similarly, the positions of the SARS CoV-2 N and S-proteins and peptides could be re-arranged or SARS CoV-2 M or E proteins or peptides could be used instead of, or in addition, to the N and S proteins. Also, as in the device of FIG. 1, SARS CoV-2 N and S-proteins and peptides (or any other SARS CoV-2 viral protein or peptide) could be used as both the labelled, mobilizable component and as the immobilized agent present on the test lines in a double antigen “sandwich” style assay.

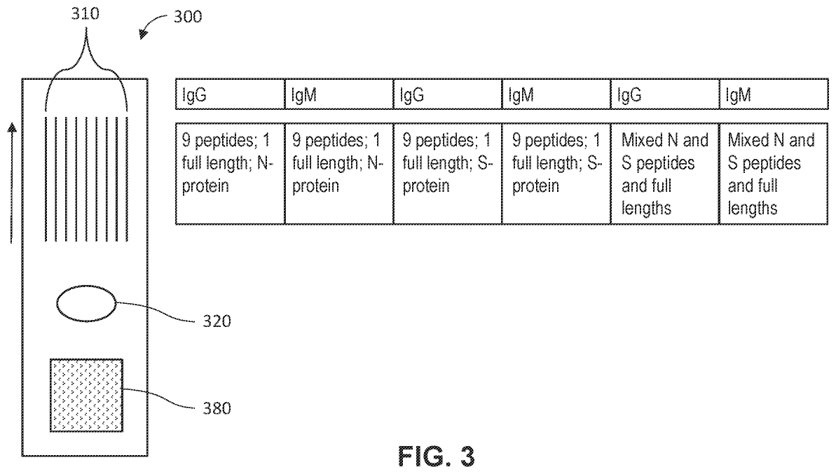

In some embodiments, the devices of the present technology comprise a plurality of parallel flow paths as shown in FIG. 3 which illustrates a top view of a unidirectional test device 300 with a plurality of parallel flow paths 310. In this exemplary configuration, the device comprises a sample receiving zone 320 and the cassette detects either human IgG or IgM antibodies for multiple SARS CoV-2 N peptides and full length N protein and/or SARS CoV-2 S peptides and/or full length S protein (as shown in the table included in the drawing). This embodiment also includes a barcode 380 for unique identification of the device. As described above, additional arrangements of the elements provided in FIG. 3, such as “antibody capture” and double antigen “sandwich” assays, are compatible with the multiple, parallel flow path devices. The configuration shown in FIG. 3 provides an example, and the other configurations and arrangements are also encompassed by this disclosure.

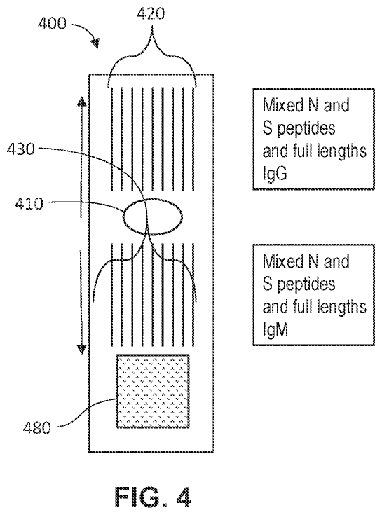

In some embodiments, the devices of the present technology comprise a plurality of bidirectional, parallel flow paths as shown in FIG. 4 which illustrates a top view of a bidirectional test device 400 with centrally located sample receiving zone 410 and a plurality of parallel, bidirectional flow paths 420 and 430. This embodiment also includes a barcode 480 for unique identification of the device. In this exemplary configuration, the cassette detects human IgG and IgM antibodies for multiple SARS CoV-2 N and S peptides and full length proteins. As described above, additional arrangements of the elements provided in FIG. 4, such as “antibody capture” and double antigen “sandwich” assays, are compatible with the multiple, bidirectional, parallel flow path devices. The configuration shown in FIG. 4 provides an example, and the other configurations and arrangements are also encompassed by this disclosure.

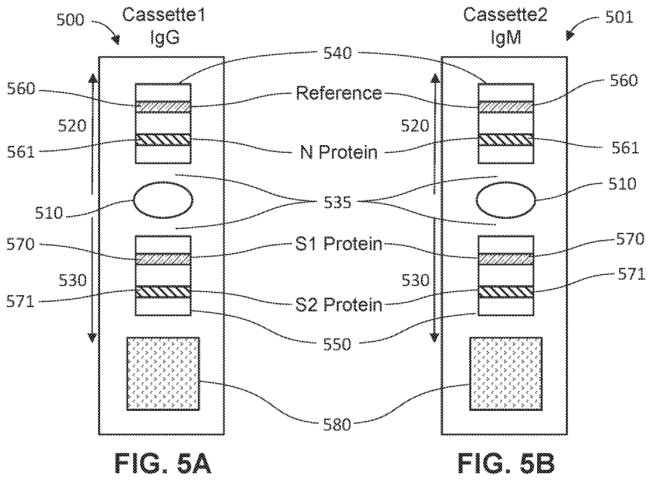

Other embodiments of the present technology are shown in FIGS. 5A and 5B, which illustrate top views of bidirectional test devices 500 (Cassette 1, IgG) and 501 (Cassette 2, IgM) with centrally located sample receiving zones 510 and bilateral fluid flow paths 520 and 530. Positions of optical windows 540 and 550 for inspection of reference line 560 and test lines 561, 570 and 571 are also shown. This embodiment also includes a barcode 580 for unique identification of the device.

In this exemplary “antibody capture” configuration, cassette 1 detects human IgG antibodies specific for SARS CoV-2 N, S1 and S2 proteins, while cassette 2 detects human IgM antibodies specific for SARS CoV-2 N, S1 and S2 proteins. S1 protein and S2 protein refer to subunit 1 and subunit 2, respectively, of SARS CoV-2 spike glycoprotein (S), a main target for neutralization antibody, to bind its receptor, and mediate membrane fusion and virus entry. Each monomer of trimeric S protein is about 180 kDa, and contains two subunits, S1 and S2, mediating attachment and membrane fusion, respectively.

Accordingly, analysis of a sample by cassette 1 will determine whether the sample contains any, or all, of human IgG specific for SARS CoV-2 N-protein, human IgG specific for SARS CoV-2 S1-protein, and human IgG specific for SARS CoV-2 S2-protein.

Correspondingly, analysis of a sample by cassette 2 will determine whether the sample contains any, or all, of human IgM specific for SARS CoV-2 N-protein, human IgM specific for SARS CoV-2 S1-protein, and human IgM specific for SARS CoV-2 S2-protein.

Importantly, analysis of three structural proteins, N, S1 and S2, in parallel, increases specificity of the assay, decreases false positives, and allows for increased assay sensitivity.

Additionally, analysis of a sample for N, S1 and S2 specific antibodies, in parallel, provides valuable information related to whether a tested individual has been subject to a prior natural SARS CoV-2 infection or has been previously vaccinated for SARS CoV-2 (See, https://www.cdc.gov/coronavirus/2019-ncov/lab/resources/antibody-tests-guidelines.html). Specifically, vaccines that have received emergency use authorization (EUA) are designed to elicit antibodies against the SARS CoV-2 S protein. Therefore, if a serological assay detects S1 and/or S2 specific antibodies but no N protein specific antibodies in a sample, this indicates the sample is from an individual who has been previously vaccinated for SARS CoV-2 and who has not been subject to prior natural infection by the SARS CoV-2 virus. If a serological assay detects antibodies specific for N proteins in a sample, this indicates that the sample is from an individual who has had a prior natural SARS CoV-2 infection. Accordingly, serological analysis of samples with the devices described herein, such as the devices of FIGS. 5A and 5B, provide valuable details related to vaccination and infection status of tested samples.

In this embodiment, specificity for human IgG or IgM is achieved by the inclusion of a non-human anti human IgG or IgM antibody in the device. The non-human anti human IgG or IgM antibody can be included, in a mobilizable fashion, in a sample pad (not shown) wherein the sample pad is located in a sample label zone 535 downstream of the sample receiving zone 510 and upstream of the reference line 560 and test lines 561, 570 and 571. In the embodiment of FIG. 5A, the sample pad in sample label zone 535 of cassette 1 comprises mobilizable, non-human anti human IgG. In the embodiment of FIG. 5B, the sample pad in sample label zone 535 of cassette 2 comprises mobilizable, non-human anti human IgM. In some embodiments, the non-human anti human IgG or IgM antibody can also be present in a reaction mixture or excipient mixture added to the sample prior to its addition to the sample receiving zone.

In some embodiments, the non-human anti human IgG or IgM is a rabbit antibody specific for human IgG or IgM (rb-α-IgGh or rb-α-IgMh). The non-human anti human IgG or IgM antibody may also include a detectable label for visualization purposes. For example, the non-human anti human antibody may include a detectable bead, such as a europium bead or a gold particle.

Accordingly, the non-human anti human antibody, in some embodiments, is a goat or rabbit antibody, specific to human IgG or IgM, conjugated with a detectable label, such as a europium bead or a gold particle. The detectable non-human anti human IgG and/or IgM antibody is referred to herein as a detectable reagent.

Also in this embodiment, specificity for SARS CoV-2 N, S1 and S2-proteins is conferred by the presence of SARS CoV-2 N, S1 or S2-proteins or peptides immobilized on the test lines 561 (N protein), 570 (S1 protein) and 571 (S2 protein) of the device. For cassette 1, detection of a signal at test line 561 indicates the sample contains human IgG antibodies specific for SARS CoV-2 N-protein, detection of a signal at test line 570 indicates the sample contains human IgG antibodies specific to SARS CoV-2 S1 protein, and detection of a signal at test line 571 indicates the sample contains human IgG antibodies specific to SARS CoV-2 S2 protein. For cassette 2, detection of a signal at test line 561 indicates the sample contains human IgM antibodies specific for SARS CoV-2 N-protein, detection of a signal at test line 570 indicates the sample contains human IgM antibodies specific to SARS CoV-2 S1 protein, and detection of a signal at test line 571 indicates the sample contains human IgM antibodies specific to SARS CoV-2 S2 protein. For both cassettes, detection of a signal at reference line 560 indicates that the test has performed properly.

The components of the device provided in FIG. 5 exemplify one possible arrangement of the provided reagents and devices described herein. However, this arrangement is not to be construed as limiting, only as exemplary. Alternative arrangements of the reagents, some of which are described below, may be apparent to the skilled artisan and are encompassed by this disclosure.

For example, in FIG. 5, the positions of the immobilized SARS CoV-2 N, S1 and S2 proteins could be swapped. Alternatively, different SARS CoV-2 proteins or peptides, such as M and/or E peptides, could be immobilized. Alternatively, cassette 1 could be made specific for IgM with cassette 2 being specific for IgG without interfering with the performance of the device. Also, the mobilizable non-human antibodies specific for human IgG or IgM could instead be immobilized on the test lines (561, 570 and 571) while the SARS CoV-2 N, S1 and/or S2-proteins and peptides could be labeled, mobilizable and present on a sample pad in the sample label zone 535.

In addition, in FIG. 5, SARS CoV-2 N, S1 and/or S2 proteins and peptides (or any other SARS CoV-2 viral protein or peptide) could serve as both the labeled, mobilizable element and the capture agent immobilized at the test lines (561, 570 and 571) in a double antigen “sandwich” assay consistent with the device provided in FIG. 1. In this arrangement, any human or non-human antibody in a test sample that is specific to SAR COV-2 N, S1 and/or S2 protein would first bind the labeled, mobilizable N, S1 or S2 protein or peptide to form a complex. The detectable labeled, mobilizable N, S1 or S2 protein or peptides are referred to herein as detectable reagents. In an embodiment, the detectable reagent is deposited in a sample label zone 535 and once bound to any IgG or IgM present in the test sample, the complex would move downstream where it would be immobilized and visualized by binding another N, S1 or S2 protein or peptide that is immobilized on the test strip on the test lines (561, 570 and 571). In this potential configuration, immunoglobulin subtype (IgG versus IgM) is not determined. This “sandwich” arrangement, however, demonstrates another alternative configuration of the devices described herein. In another embodiment, the detectable reagent is provided in a vial or container separate from the immunoassay device, such as in a reagent mixture or excipient mixture, that is combined with the test sample prior to its placement on the sample receiving zone.

The potential rearrangements would not interfere with the performance of the device and would still permit analysis of a sample for the presence of antibodies specific for SARS CoV-2 proteins and peptides, such as N, S1, S2, M and/or E-proteins and peptides. Accordingly, these alternative arrangements of components are included, either explicitly or implicitly, in this disclosure.

Additional details for immunoassay designs, configurations, and materials for fabricating the devices are described in U.S. Published Application Nos. 2017/0059566, US 2019/0118181, and U.S. 2018/0229232, each incorporated by reference herein in its entirety.

Instruments for interrogating the test lines on the immunoassay device and for analyzing the image and data from the interrogation are described, for example, in U.S. Pat. Nos. 9,207,181 and 9,989,466, incorporated by reference herein. Methods for transmitting data from such instruments and for conducting surveillance of infections based on data obtained from such instructions are described, for example, in U.S. Pat. No. 10,541,056, incorporated by reference herein. As can be appreciated, the instruments comprise a processor with an algorithm for analysis of the signal produced at the test line(s) in the one or more capture zones. The algorithm in some embodiments, will inspect the data from the instrument to determine presence or absence of signal at the N-test line(s) and/or the S-test line(s) in a desired sequence for clinical purposes. For example, in some cases it may be clinically useful to ascertain if immunoglobulins are present at both the N-test line and the S-test line, or in other cases it may be desired to know only if a person has S-protein specific immunoglobulins. The algorithm can be written to query and assess the data according to the desired clinical information. By way of another example, for immunoassay devices that comprise multiple fluid flow channels each with a test line, for example 10 test lines each in a discrete fluid flow channel, where each test line has an immobilized N- or S-peptide or protein from SARS CoV-2, the algorithm can report a positive or negative result based on inspection of 2, 3, 4, 5, 6, 7, 8, 9, 10, 11, and/or 12 peptides are present or based on whether a particular combination of peptides or proteins is/are present.

### III. Kits and Methods of Use

Severe acute respiratory syndrome coronavirus 2 (SARS CoV-2) is the virus strain that causes coronavirus disease 2019 (COVID-19), a respiratory illness. The test strips described herein provides a sensitive and specific diagnostic method for the detection of human antibodies specific for SARS CoV-2, e.g. at early times after infection, and provides a means to stage the infection as early or late stage. The test strips described herein provide methods for determining the presence or absence of human IgG and/or IgM specific for COV-2. The methods and devices provided herein are also specific for antibodies to at least two different SARS CoV-2 proteins, SARS CoV-2 N and S-proteins.

Kits comprising one or more of the devices provided herein, along with instructions for using the devices in methods of detecting human antibodies specific SARS CoV-2 for are also provided.

Accordingly, methods using the devices provided herein overcome several problems associated with other lateral flow strip tests. For example, other strip tests may exhibit false positive results due to due to interference. This problem is overcome with the methods, devices and kits described herein because the presently described devices and methods provide measurement of two independent events (i.e., presence of 2 different proteins/peptides, SARS CoV-2 N-protein and SARS CoV-2 S-protein). Accordingly, in some methods of the present technology, a test only provides a positive result if human antibodies to both SARS CoV-2 N-protein and SARS CoV-2 S-proteins are detected. This format helps to overcome any false positive issues associated with other SARS CoV-2 lateral flow immunoassays.

Another problem exhibited by other strip tests is a false negative result that occurs due to a low viral titer. The devices and methods described herein can be overcome this challenge by being more sensitive and amenable to objective instrument read-out. The requirement to determine 2 independent positive events (i.e., presence of 2 different proteins/peptides, SARS CoV-2 N-protein and SARS CoV-2 S-protein) provides higher specificity and thereby allows decrease of cutoff level for positive results and reduction of false negative results.

Another problem associated with other serological tests is related to disease progression and the possibility that no human IgG has yet been generated in response to the viral infection. This may also be the case, and result in false negative results during an early infection window where low or no viral titer is present. In these cases, only IgM may be present, and any human antibody specific for SARS CoV-2 may be present in low amounts. The devices, methods and kits provided herein address this problem by measuring both IgG and IgM, for example in a bidirectional assay and by measuring two independent events (SARS CoV-2 N-protein and SARS CoV-2 S-protein) each of which provides improved specificity (of particular importance for IgM).

Another problem associated with other serological test is that different individual human's exhibit varying responses to SARS CoV-2 N-protein and SARS CoV-2 S-protein. This challenge is again overcome by the methods, devices and kits provided herein because the methods provided herein measure both to SARS CoV-2 N and S-protein with separate titers on a single test strip. Accordingly, the methods provided herein are sensitive to infection duration changes for IgG and IgM ratios and for SARS CoV-2 N and/or S-protein response.

Another problem associated with other serological tests related to SARS CoV-2 is that the test is not able to detect reflect immunprotection based on the presence of neutralizing antibodies. The methods, devices and kits provided herein address this challenge by measuring multiple proteins and peptides (SARS CoV-2 N and S-proteins and peptides) in order to measure both overall response and to detect the presence of neutralizing abs. This allows the presently described tests to be able to answer at least two questions: (i) whether an individual has been infected and (ii) whether an individual is protected from future infections. Along the same lines, the presently described tests are also able to determine the stage of an infection and whether an individual is still infectious based on the IgM/IgG ratio. This also allows the devices, methods and kits described herein to ascertain whether a protective titer has been achieved in a given individual by measuring titers quantitatively.

The methods, devices and kits described herein also provide advantages with respect to reduced false positive and false negative results due to improved accuracy facilitated by the use of an instrument, such as an optical reader, to analyze the results.

In some embodiments, the devices, methods and kits provided herein can be provided in a manner in which the test can be performed at home, with or without a prescription from a health care professional. In the burgeoning area of telemedicine, it has become increasingly desirable to take advantage of the almost universal availability of electronic appliances that may have wireless network access, sensors, and that may also include increasingly higher computational capabilities. Further, mobile computer devices include image-capturing capabilities with increased resolution and higher capability for digital processing (e.g., spatial filtering and adjustment, and spectral filtering). Moreover, some applications of remote measurement of immunoassays designed for the detection of chemical and biological agents or pathogens may include security tests and screening (e.g., at airports, police, and military checkpoints), or environmental analysis and monitoring (e.g., air pollution, contamination of water ways and reservoirs for disease control or agricultural production, and the like).

Embodiments consistent with the present disclosure take advantage of the high image-capturing and processing capabilities of current consumer appliances to provide simple yet accurate diagnostic procedures for selected diseases (e.g., legionella, influenza, Ebola, Lyme disease, and the like). The types of tests consistent with embodiments in the present disclosure may include any type of spectroscopic analysis of test assays using electromagnetic radiation, such as, without limitation, absorption spectroscopy (ultra-violet, visible, or infrared), including reflectance or transmittance spectroscopy, or emission spectroscopy, including fluorescence and luminescence spectroscopy, Raman spectroscopy, and any type of radiation scattering. Moreover, embodiments as disclosed herein may further exploit the networking capabilities of such appliances to enhance the processing, cataloging, regulating, and cross-referencing capabilities of each test by using cloud-computing solutions. Accordingly, in some embodiments, a high quality (e.g., high spatial and spectral resolution) image, sequence of images, or video, or a processed version of them is uploaded to a remote server that can perform massively parallel computations to provide, in a reduced time, a diagnostic result. Such analyzed material may be processed immediately, at a later date/time, and/or may be compared to previously collected materials to determine differences over time, e.g., a time evolution of the analyte across a test band. Such analyzed material may also, after user de-identification, be used for analyses in the interest of public health, or to provide additional benefits to the user of the test by cross-referencing the results to others with specific criteria, e.g., age group, gender, geographic location, pathogen characteristics, and the like.

The subject system provides several advantages, including the ability for a user to quickly learn whether a disease is present or latent, mild or severe, without the need to access specialized personnel, or a complex machine or instrument.

In some instances, the devices, methods and kits of the present technology include additional materials related to preparation of a liquid sample, such as blood from a finger prick. US Patent Publication No. US2018/0136194 (hereby incorporated by reference in its entirety) is related to devices for whole blood separation and provides additional details related to the same.

According to some embodiments of the present disclosure, a kit can include: a container including a reservoir for receiving a solution; a sample device including: an inflow chamber for receiving a liquid sample; a mixing chamber; a pad between the inflow chamber and the mixing chamber, the pad optionally including a processing reagent such as a red blood cell agglomerating substance; an outflow port adjacent to the mixing chamber; and an interface for sealably connecting the sample device to the container such that the reservoir is in fluid communication with the mixing chamber.

The inflow chamber can include a capillary tube. The sample device further can include a filter between the mixing chamber and the outflow port. The kit can further include a solution within the reservoir.

According to some embodiments of the present disclosure, a method can include: with an inflow chamber of a sample device, receiving a liquid sample; facilitating separation of a first portion of the liquid sample from a second portion of the liquid sample by retaining the second portion at a pad, the pad optionally including a processing reagent such as red blood cell agglomerating substance when the liquid sample is, for example, blood; facilitating flow of the first portion of the liquid sample through the pad and to a mixing chamber; sealably connecting the sample device to a container including a reservoir containing a buffer solution; mixing the first portion of the liquid sample with the buffer solution to create a mixture; and dispensing at least some of the mixture from the mixing chamber and through an outflow port of the sample device.

### IV. Examples

The following examples are illustrative in nature and are in no way intended to be limiting.

### Example 1

### Antibody Capture Test Strip Assay for Detecting Presence of Human IgG Antibodies Against SARS COV-2 N and/or SARS COV-2 S-Protein

Analysis of a sample, such as liquid blood, for the presence of α-SARS CoV-2h IgG specific for SARS CoV-2 N or S-protein is performed with a device comprising a nitrocellulose lateral flow strip test. The nitrocellulose strip test includes test lines in a capture zone comprising immobilized full length SARS CoV-2 N or S protein or peptides as a capture reagent. The strip test further includes a sample pad containing a mobilizable rabbit antibody specific for human IgG (rb-a-IgGh) conjugated to europium beads.

### Example 2

### Antibody Capture Test Strip Assay with Premix for Detecting Presence of Human Igg Antibodies Against SARS COV-2 N and/or SARS COV-2 S-Proteins

Analysis of a sample, such as liquid blood, for the presence of α-SARS CoV-2h IgG and/or α-SARS CoV-2h IgM is performed with a device comprising a nitrocellulose lateral flow strip test. The nitrocellulose strip test includes test lines in a capture zone comprising immobilized full length SARS CoV-2 N or S protein or peptides as a capture reagent. The method for using the device further includes adding a reaction mixture to the sample prior to addition to strip, wherein the reaction mixture contains a rabbit antibody specific for human IgG (rb-α-IgGh) conjugated to gold beads.

### Example 3

### Double Antigen Sandwich Test Strip Assay for Detecting Presence of Human Antibodies Against SARS COV-2 N and/or SARS COV-2 S Proteins

Analysis of a sample, such as liquid blood, for the presence of human α-SARS CoV-2 antibodies is performed with a device comprising a nitrocellulose lateral flow strip test. The nitrocellulose strip test includes test lines in a capture zone comprising immobilized full length SARS CoV-2 N or S protein or peptides as a capture reagent. The strip test further includes a sample pad containing a mobilizable, full length SARS CoV-2 N or S protein or peptides conjugated to europium beads.

### Example 4

### Double Antigen Sandwich Test Strip Assay with Premix for Detecting Presence of Human Antibodies Against SARS COV-2 N and/or SARS COV-2 S Proteins

Analysis of a sample, such as liquid blood, for the presence of human α-SARS CoV-2 antibodies is performed with a device comprising a nitrocellulose lateral flow strip test. The nitrocellulose strip test includes test lines in a capture zone comprising immobilized full length SARS CoV-2 N or S protein or peptides as a capture reagent. The method for using the device further includes adding a reaction mixture to the sample prior to addition to strip, wherein the reaction mixture contains full length SARS CoV-2 N or S protein or peptides conjugated to europium beads.

### Example 5

### Antibody Capture Test Strip Assay for Detecting Presence of Human Igg Antibodies Against SARS COV-2 N, S1 and/or S2 Protein

Analysis of a sample, such as liquid blood, for the presence of α-SARS CoV-2h IgG specific for SARS CoV-2N, S1 and/or S2-protein is performed with a device comprising a nitrocellulose lateral flow strip test. The lateral flow strip test comprises a single, central sample receiving zone and two, bi-lateral, fluid flow paths. The nitrocellulose strip test includes a test line in a first capture zone of the first fluid flow path comprising immobilized full length SARS CoV-2 N protein. The nitrocellulose strip test also includes two test lines in a second capture zone of the second fluid flow path comprising immobilized full length SARS CoV-2 S1 and S2 protein, respectively, as capture reagents. The strip test further includes a sample pad in each fluid flow path containing a mobilizable rabbit antibody specific for human IgG (rb-α-IgGh) conjugated to europium beads.

### Example 6

### Antibody Capture Test Strip Assay for Detecting Presence of Human IGM Antibodies Against SARS COV-2 N, S1 and/or S2 Protein

Analysis of a sample, such as liquid blood, for the presence of α-SARS CoV-2h IgM specific for SARS CoV-2N, S1 and/or S2-protein is performed with a device comprising a nitrocellulose lateral flow strip test. The lateral flow strip test comprises a single, central sample receiving zone and two, bi-lateral, fluid flow paths. The nitrocellulose strip test includes a test line in a first capture zone of the first fluid flow path comprising immobilized full length SARS CoV-2 N protein. The nitrocellulose strip test also includes two test lines in a second capture zone of the second fluid flow path comprising immobilized full length SARS CoV-2 S1 and S2 protein, respectively, as capture reagents. The strip test further includes a sample pad in each fluid flow path containing a mobilizable rabbit antibody specific for human IgM (rb-α-IgMh) conjugated to europium beads.

### Example 7

### Antibody Capture Test Strip Assay for Distinguishing Vaccination from Prior Natural SARS COV-2 Infection

Serological analysis of samples with the devices described herein, such as the devices of FIGS. 5A and 5B, provide valuable details related to vaccination and infection status of tested samples. For example, serological analysis of a sample for antibodies against N, S1 and S2 proteins/peptides, in parallel, provides details related to whether a tested individual has been subject to a prior natural SARS CoV-2 infection or has been previously vaccinated for SARS CoV-2. Specifically, vaccines that have received emergency use authorization (EUA), including Moderna and Pfizer vaccines, are designed to elicit antibodies against the SARS CoV-2 S protein. Therefore, if a serological assay detects S1 and/or S2 specific antibodies but no N protein specific antibodies in a sample, this indicates the sample is from an individual who has been previously vaccinated for SARS CoV-2 and who has not been subject to prior natural infection by the SARS CoV-2 virus. Alternatively, if a serological assay detects antibodies specific for N proteins in a sample, this indicates that the sample is from an individual who has had a prior natural SARS CoV-2 infection.

The study described below examines the ability of the devices and methods described herein to distinguish samples from vaccinated individuals with no prior natural SARS CoV-2 infection and samples from individuals with prior natural SARS CoV-2 infection.

To further understand the production of neutralizing antibodies in vaccinated individuals, a prospective study was conducted with Institutional Review Board (IRB) approval, to enroll subjects who were willing to donate a pre-vaccination and multiple post vaccination blood specimens. Donors were divided into 2 groups: those with evidence of infection prior to vaccination (positive COVID-19 antigen or serology test result) and those with no prior evidence of infection. Donors were immunized using either a vaccine available from Moderna or from Pfizer/BioNTech. Blood specimens were taken every 1 to 2 weeks post vaccination and the regimen after the second vaccination was followed out at least 6 months, with monthly blood draws. Data provided herein is related to samples collected post vaccination and out to approximately 2 weeks after the second vaccination. All specimens were tested using the devices described herein, such as the device of FIG. 5A.

The lateral flow rapid test immunoassay device used for this analysis, such as the device shown in FIG. 5A, is for the qualitative detection and of Immunoglobulin G (IgG) antibodies to SARS CoV-2 from finger stick whole blood, venous whole blood, serum, or plasma samples. The assay is designed to semi-quantitatively detect and differentiate between SARS CoV-2 N, S1 and S2 IgG responses. The recombinant N protein consists of 425 amino acids with a predicted molecular mass of 46.45 kDa (SEQ ID NO: 25). A DNA sequence encoding the SARS-CoV-2 (2019-nCoV) N (1-419) was expressed in E. coli. The recombinant SARS-CoV-2 (2019-nCoV) Spike Protein (S1 subunit) consists of 681 amino acids with a predicted molecular mass of 76.5 kDa (SEQ ID NO: 26). A DNA sequence encoding the SARS-CoV-2 (2019-nCoV) spike protein S1 Subunit (YP_009724390.1) (Val16-Arg685) was expressed in HEK293 cells. The recombinant SARS-CoV-2 (2019-nCoV) Spike Protein (S2 ECD subunit) consists of 539 amino acids with a predicted molecular mass of 59.37 kDa (SEQ ID NO: 27). A DNA sequence encoding the SARS-CoV-2 (2019-nCoV) spike protein S2 ECD Subunit was expressed in Baculovirus-Insect cells.

To perform the lateral flow test with whole blood samples from a finger stick, a 20-25 μL specimen is collected into a small disposable capillary device. This specimen is diluted into a pre-filled vial that contains reagent solution, such as a buffered saline solution. 100 μL of the diluted specimen is applied to the sample well located in the middle of the test device. For serum and plasma sample types, 10 μL of the specimen is diluted into a pre-filled vial that contains reagent solution. For all sample types, the sample flows into the sample receiving zone, then to the sample label zones, prior to being delivered to first and second capture zones which contain test lines for S1, S2, and N protein specific antibody binding, as well as a reference line that serves a control for proper device performance. SARS CoV-2 IgG antibodies (if present in the sample), bind to fluorescent beads containing anti-human IgG antibodies in the sample label zone and will subsequently be immobilized by Nucleocapsid Protein (N) antigen, Spike S1 and S2 antigens on test lines on the nitrocellulose in the capture zones.

After 15 minutes, results are read with an automated optical reader device, such as the Sofia 2 instrument available from Quidel Corporation (www.quidel.com/immunoassays/sofia-tests-kits/sofia-2-analyzer) to process and report the test results. A positive or negative result is reported for each of the test lines (N, S1 and S2). The qualitative values for each antigen are based on signal to cutoff values (S/Co) where an S/Co≥1 is positive and an S/Co<1 is negative.

Sample Enrollment and Collection:

A multi-center, IRB approved, prospective study was designed to procure specimens prior to and after the first and second COVID-19 immunizations. Enrolled subjects were either previously infected with COVID-19 or had not been infected previous to the first vaccine inoculation. Other inclusion criteria included any male or female subject 18 years or older, subjects must have received the first COVID-19 vaccination 7 days or less prior to the first blood draw and subjects were required to perform the second vaccination according to the manufacturer's requirement. Following consent, demographics, symptoms and health history were collected from each subject. Matched finger stick whole blood, venous whole blood, plasma and serum (separator tube) specimens were collected from each subject at each visit. The blood specimens were processed and tested the same day as collected using lateral flow devices described herein, such as the device of FIG. 5A.

A total of 92 subjects were enrolled in the study. 52 subjects had received their first dose of the Moderna vaccine and 40 subjects had received their first dose of the Pfizer vaccine. 50 subjects had received their second dose of Moderna and 32 subjects had received their second dose of the Pfizer vaccine. The average number of days between the first and second doses varied between the Moderna and Pfizer vaccines due to the manufacturer recommendations as well as expected logistics (scheduling, weather delays, vaccine supply) involved in a second visit. The average time between doses for subjects receiving the Moderna vaccine was 32 days, approximately 4 days beyond the manufacturer recommendation. The average time between doses for subjects receiving the Pfizer vaccine was 21 days, which is the Pfizer recommendation. Given that the immune response to the first and second doses varies and that the CDC recommendation for protection is 2 weeks past the second dose for both vaccines, subjects that were at least 2 weeks past the second dose were used to standardize the data. 23 subjects that had received the Moderna vaccine were 2 weeks past the second dose and 13 subjects that had received the Pfizer vaccine were 2 weeks past the second dose. Demographic data is summarized in Table 2.

TABLE 2
SUBJECT DEMOGRAPHICS

| | Moderna | Pfizer | Total |
|---|---|---|---|
| Subjects received 1st Dose | 52 | 40 | 92 |
| Subjects received 2nd Dose | 50 | 32 | 82 |
| Subjects >2 weeks past | 50 | 30 | 80 |
| second dose | | | |
| Average Days Between | 32 | 21 | NA |
| Doses | | | |
| Gender Male | 28 | 14 | 42 |
| Gender Female | 24 | 26 | 50 |
| Age Range | 19-79 | 19-83 | 19-83 |
| Median Age | 40 | 40 | 40 |
| Subjects with Prior Natural | 4 | 2 | 6 |
| Infection | | | |
| Subjects with no Prior | 48 | 38 | 86 |
| Infection | | | |

Lateral flow devices, such as the device of FIG. 5A, were used to analyze a total of 816 COVID-19 negative serum and plasma samples that were collected prior to the COVID-19 pandemic. The specificity was 98.4% (803/816) for N, 99.9 (815/816) for S1 and 99.0% (808/816) for S2 (Table 3). In addition, a panel of multiple donors (99) from 17 non-COVID-19 diseased state serum and plasma specimens that included seasonal coronavirus was evaluated (Table 3). The specificity of the diseased state specimens was 98.0% (97/99) for N, 100.% (99/99) for S1 and 99.0% for S2 (98/99).

TABLE 3
N, S1 AND S2 SPECIFICITY WITH RETROSPECTIVE
PRE-COVID-19 AND DISEASE STATE SPECIMENS

| Specimen | Protein | Tested | Negative | Positive | Specificity |
|---|---|---|---|---|---|
| Pre-COVID 19 | N | 816 | 803 | 13 | 98.4% |
| Negative | | | | | |
| Pre-COVID 19 | S1 | 816 | 815 | 1 | 99.9% |
| Negative | | | | | |
| Pre-COVID 19 | S2 | 816 | 808 | 8 | 99.0% |
| Negative | | | | | |
| Disease State | N | 99 | 97 | 2 | 98.4% |
| Disease State | S1 | 99 | 99 | 0 | 99.9% |
| Disease State | S2 | 99 | 98 | 1 | 99.0% |

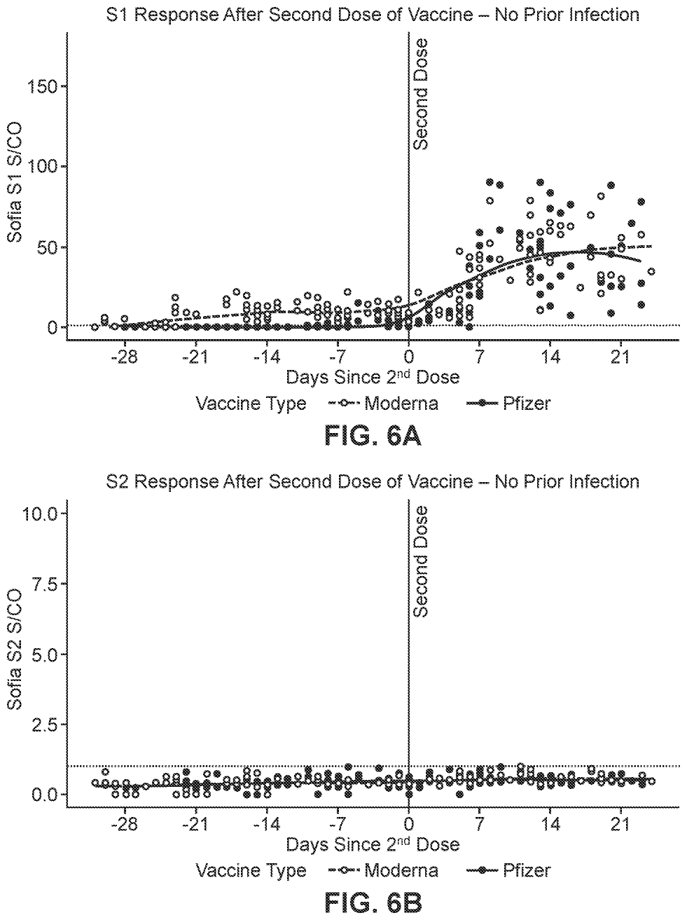

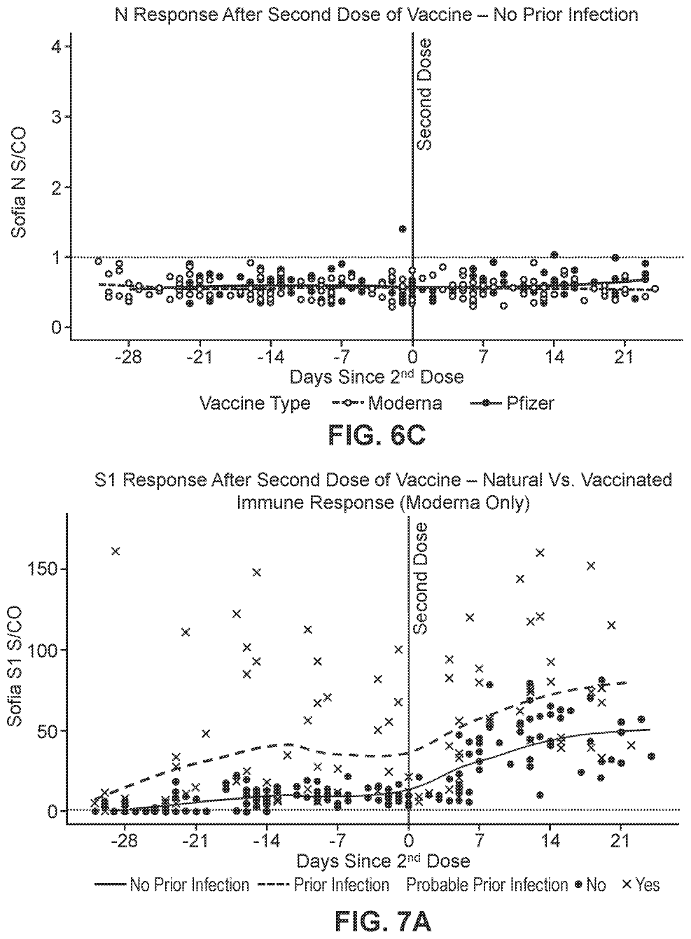

Lateral flow test devices, such as the device of FIG. 5A, were also used to monitor the IgG immune response to N, S1 and S2 proteins of vaccinated subjects (Moderna, n=50 and Pfizer, n=30) that had no prior infection with SARS CoV-2. Semi-quantitative signal to cutoff (S/CO) results for S1, S2 and N from finger stick whole blood, from subjects a minimum of 2 weeks past the second dose are plotted over time (weeks) adjusted to the week that the 2nd vaccine dose was administered (FIG. 6). The primary IgG immune response for both vaccines was against S1 in subjects that had no known previous exposure to SARS CoV-2. There was minimal IgG antibody response observed against the S2 and N proteins (Sofia S2 and N S/CO<1.0). Most subjects receiving the Moderna vaccine had an immune response to S1 (S/CO>1.0) approximately 2 weeks (13 days) after the 1st vaccine dose. Most subjects that received the Pfizer vaccine had an S1 immune response (S/CO>1.0) approximately 3 weeks (20 days) after the first vaccine dose. With data from both vaccine groups normalized to the second vaccine dose, the average antibody response to S1 is similar approximately 2 weeks after the second dose.

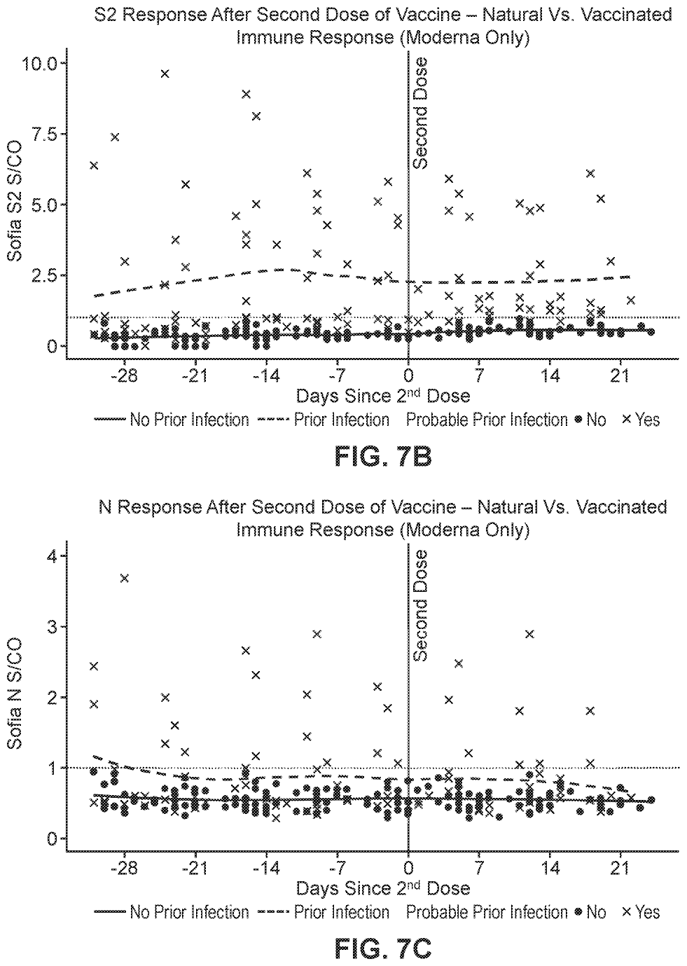

Lateral flow test devices, such as the device of FIG. 5A, were also used to monitor the IgG immune response to N, S1 and S2 proteins of vaccinated subjects (Moderna=4, Pfizer=2) previously exposed to SARS CoV-2 (FIG. 7). The IgG immune response in prior natural SARS CoV-2 infected samples was notably different than the immune response from subjects that had not been previously exposed. Specifically, the pre-exposed subjects showed a baseline immune response to all 3 antigens compared to subjects from the non-exposed group. Following the first vaccination, the measured S1 IgG response for the pre-exposed group was rapid and significantly high (S1 S/CO=100) compared to the non-exposed subjects (S1 S/CO=15). The S1 IgG increase was seen rapidly after the 1st vaccination compared to the non-exposed subjects. Following the 2nd vaccination, the pre-exposed subject S1 response did not seem to reach a similarly high level that was observed after the first vaccination dose.

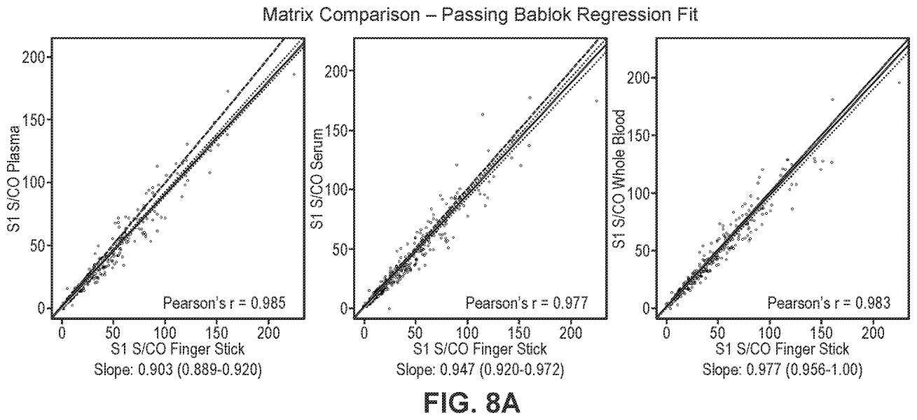

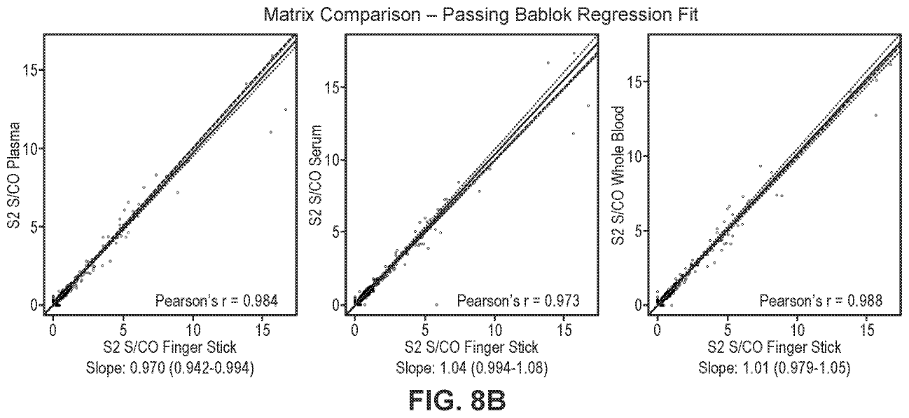

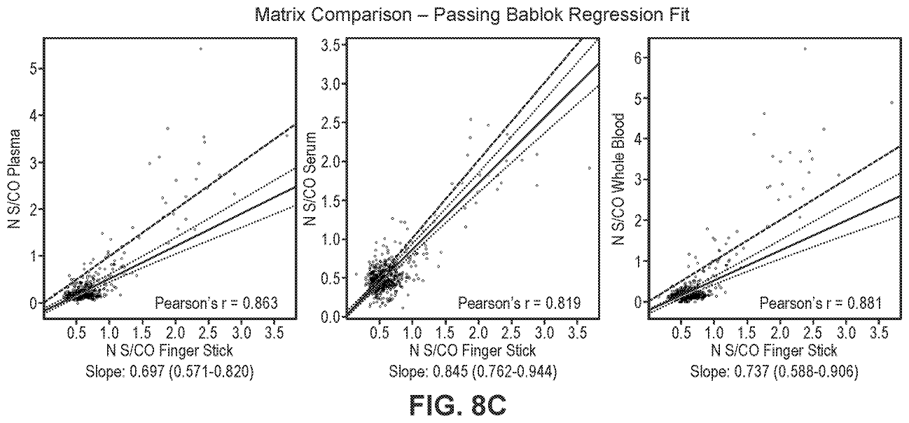

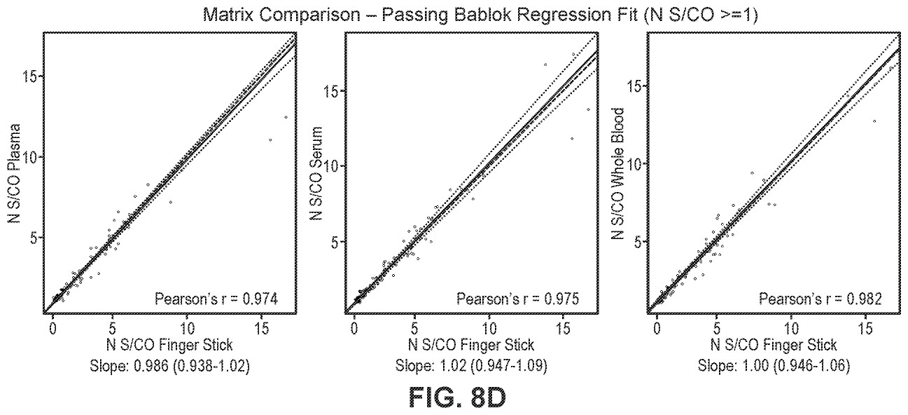

The lateral flow devices described herein are designed to be used with finger stick whole blood, venous whole blood, serum, and plasma samples. In near patient settings, the finger stick whole blood sample is advantageous due to a simple and short collection process that only requires 20-25 μl whole blood that may be collected in a small disposable plastic capillary tube. The IgG response for S1, S2 and N proteins was compared for the four sample types using data from each, over time, to determine if sample type is associated with any sample diagnostic difference. Results of this analysis for N protein are shown in Table 4. Statistical analysis for detection of S1, S2 and N IgG antibodies from finger stick whole blood, venous whole blood, serum, and plasma samples are provided in FIG. 8A-D. The results demonstrate there is no significant difference in diagnostic result associated with the different blood sample types.

TABLE 4
COMPARISON OF FINGER STICK
TO OTHER SAMPLE TYPES (N)

| Sample | Finger stick | | |
|---|---|---|---|
| Type | % Agreement | 95% CI | |
| Plasma | 96.4% (532/552) | 94.5% | 97.6% |
| Serum | 95.6% (525/549) | 93.6% | 97.0% |
| Venous | 97.1% (536/552) | 95.3% | 98.2% |
| Whole blood | | | |

In conclusion, the results provided herein demonstrate that the currently disclosed lateral flow strip test immunoassay devices can be used to separately detect SARS CoV-2 S1, S2 and N IgG immune responses in tested samples. IgG immune responses to S1 were detected in samples from vaccinated individuals who had been previously exposed/infected with SARS CoV2 and individuals who had not been previously exposed. There was a minimal N and S2 IgG immune response in vaccinated subjects who had not been previously exposed to SARS CoV-2. This indicates that the methods and devices described herein can distinguish samples from SARS CoV-2 vaccinated individuals without prior infection from samples from prior natural SARS CoV-2 infected individuals. Moreover, this study shows that accurate results can be obtained using a capillary finger stick sample to rapidly provide results that differentiate between vaccinated subjects that had previously been diagnosed with COVID-19 and subjects had not been diagnosed with COVID-19.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. An immunoassay device for detection of an immunoglobulin for SARS CoV-2, comprising:
a sample receiving zone configured to receive a blood, plasma or serum sample,
a detectable reagent comprising
i) a SARS CoV-2 S protein or peptide and a SARS CoV-2 N protein or peptide; or
ii) a non-human anti-human IgG, a non-human antihuman IgM antibody, or both;
one or more capture zones comprising
an S-test line comprising an immobilized SARS CoV-2 S protein or peptide;
an N-test line comprising an immobilized SARS CoV-2 N-protein or peptide; or
an S-test line and an N-test line
wherein a sample deposited in the sample receiving zone is confirmed to comprise an immunoglobulin for SARS CoV-2 when the detectable reagent is detected at the S-test line, the N-test line, or both.

2. The immunoassay device of claim 1, wherein
the detectable reagent comprises a SARS CoV-2 S protein or peptide and a SARS CoV-2 N protein or peptide.

3. The immunoassay device of claim 1, wherein
the detectable reagent comprises a non-human anti-human IgG and a non-human antihuman IgM antibody.

4. The immunoassay device of claim 1, wherein the device comprises two capture zones, a first capture zone comprising the S-test line and the N-test line for capture of the detectable reagent comprising a non-human anti-human IgG, and a second capture zone comprising a second S-test line and a second N-test line for capture of the detectable reagent comprising a non-human anti-human IgM.

5. The immunoassay device of claim 1, wherein
the detectable reagent comprises a non-human anti-human IgG antibody.

6. The immunoassay device of claim 1, wherein
the detectable reagent comprises a non-human anti-human IgM antibody.

7. The device of claim 1, wherein the one or more capture zones comprises a first capture zone and a second capture zone, the first capture zone comprising the N-test line in a first fluid flow path in communication with the sample receiving zone, and the second capture zone comprising the S-test line and in a second fluid flow path in communication with the sample receiving zone.

8. The device of claim 1, wherein the detectable reagent is deposited on the device in conjunction with a test sample.

9. The device of claim 1, wherein the detectable reagent is in a reagent zone downstream of the sample receiving zone.

10. The device of claim 1, wherein the SARS CoV-2 S protein or peptide and the SARS CoV-2 N-protein or peptide on the S-test line and the N-test line, respectively, is selected from the group of sequences in Table 1 or any sequence having 99%, 98%, 97%, 96%, 95%, 94%, 93%, 92%, 91%, 90%, 89%, 88%, 87%, 86% or 85% sequence identity thereto.

11. The device of claim 1, wherein the detectable reagent comprises a SARS CoV-2 S protein or peptide and a SARS CoV-2 N-protein or peptide selected from the group of sequences in Table 1 or any sequence having 99%, 98%, 97%, 96%, 95%, 94%, 93%, 92%, 91%, 90%, 89%, 88%, 87%, 86% or 85% sequence identity thereto.

12. The device of claim 1, wherein the SARS CoV-2 S protein or peptide and the SARS CoV-2 N-protein or peptide on, respectively, the S-test line and the N-test line, is, respectively, the full length SARS CoV-2 S protein and the full length SARS CoV-2 N-protein or any sequence having 99%, 98%, 97%, 96%, 95%, 94%, 93%, 92%, 91%, 90%, 89%, 88%, 87%, 86% or 85% sequence identity thereto.

13. The device of claim 1, wherein the one or more capture zones each comprise between 2-12 test lines, each test line in a discrete fluid flow path in fluid communication with the sample receiving zone.

14. The device of claim 13, wherein each test line in the one or more capture zones comprises a different immobilized SARS CoV-2 S- and/or SARS CoV-2 N-protein or peptides.

15. The device of claim 1, wherein the device comprises 10 test lines, each test line in a discrete fluid flow path in fluid communication with the sample receiving zone.

16. The device of claim 1, wherein when the detection reagent comprises a SARS CoV-2 S- or SARS CoV-2 N-protein or peptide, the at least one capture zone comprises the same SARS CoV-2 S- or SARS CoV-2 N-protein or peptides immobilized thereon.

17. The device of claim 1, wherein the detectable reagent comprises a detection moiety selected from a chelated lanthanide or metal.

18. The device of claim 17, wherein the chelated lanthanide is europium.

19-29. (canceled)

30. A kit comprising:
one or more immunoassay devices according to claim 1;
optionally, a detection reagent;
optionally, a blood collection device; and/or
optionally, an auxiliary unit with an imaging system and electronics for transmitting an image wirelessly.

31. A method to distinguish an immunoglobulin for SARS CoV-2 as one from infection by SARS CoV-2 virus or one from a vaccination, comprising:
detecting in a blood sample antibodies against the S protein of SARS CoV-2 virus and antibodies against the N protein of SARS CoV-2 virus;
reporting one or more of:
i) the blood sample as being from an individual infected with SARS CoV-2 virus and/or previously vaccinated if antibodies against both the N-protein and the S-protein are detected;
ii) the blood sample as being from an individual with SARS CoV-2 virus if antibodies against the N-protein are detected and antibodies against the S-protein are not detected or are present but below a defined threshold;
iii) the blood sample as being from an individual previously treated with a vaccine against SARS CoV-2 virus if antibodies against the S-protein are detected and antibodies against the N-protein are not detected or are present but below a defined threshold;
iv) the blood sample as being from an individual with no immunoglobulin for SARS CoV-2 if antibodies against the S-protein and the N-protein are not detected; or
v) invalid result if a control line has no detectable control reagent.

32-44. (canceled)
